(19) United States
(12) Patent Application Publication
(10) Pub. No.: US 2001/0039275 A1
(43) Pub. Date: Nov. 8, 2001

(54) Use of 2,4-diaminothiazole derivatives

(76) Inventors: Andrew Neil Bowler, Vaerlose (DK); Preben Houlberg Olesen, Kobenhavn NV (DK); Anders Robert Sorensen, Herlev (DK); Bo Falck Hansen, Virum (DK); Helle Worsaae, Gentofte (DK); Peter Kurtzhals, Taastrup (DK)

Correspondence Address: Steve T. Zelson, Esq., Novo Nordisk of North America, Inc., Suite 6400, 405 Lexington Avenue, New York, NY, 10174-6400, US

(21) Appl. No.: 09/774,900

(22) Filed: Jan. 31, 2001

Related U.S. Application Data
(60) Provisional application No. 60/183,518, filed on Feb. 18, 2000.

(30) Foreign Application Priority Data
Feb. 4, 2000 (DK) PA 2000 00187

(51) Int. Cl. A61K 31/5377; A61K 31/497; A61K 31/506; A61K 31/4439; A61K 31/425

(52) U.S. Cl. 514/235.5; 514/252.05; 514/255.05; 514/257; 514/256; 514/370; 514/340; 544/133; 544/238; 544/333; 544/405; 546/270.7; 548/191

(57) ABSTRACT

2,4-Diaminothiazole derivatives which inhibit GSK-3 (glycogen synthase kinase-3) and which are useful for the treatment and/or prevention disorders and diseases wherein an inhibition of GSK-3 is beneficial, especially especially Alzheimer's disease, bipolar disorder, IGT (impaired glucose tolerance), Type 1 diabetes, Type 2 diabetes and obesity.

### FIELD OF THE INVENTION

The present invention relates to the use of 2,4-diaminothiazole derivatives of the general formula (I) for the preparation of pharmaceutical compositions for the treatment and/or prevention of disorders and diseases wherein an inhibition of GSK-3 (glycogen synthase kinase-3) is beneficial, especially Alzheimer's disease, bipolar disorder, IGT (impaired glucose tolerance), Type 1 diabetes, Type 2 diabetes and obesity. Some of the 2,4-diaminothiazole derivatives are novel per se and constitute a further aspect of the invention.

### BACKGROUND OF THE INVENTION

GSK-3 is a protein-serine kinase implicated in the hormonal control of several regulatory proteins. It was first discovered by virtue of its ability to phosphorylate and inactivate glycogen synthase, the regulatory enzyme of glycogen synthesis in mammals (Embi, N. et al. (1980), EUR J BIOCHEM 107, 519-527; Rylatt, D. B. et al. (1980), EUR J BIOCHEM 107, 529-537). Since then a number of other substrates have been identified, implicating the enzyme in the regulation of several physiological processes.

GSK-3 exists in two isoforms, termed GSK-3α and GSK-3β, which are derived from distinct genes and show 85% sequence identity. Unlike many protein kinases, both GSK-3 isoforms are constitutively active in resting cells, and are primarily regulated by inactivation. Thus, it has been shown that GSK-3 is inhibited by serine phosphorylation in response to insulin and growth factors such as IGF-1 and EGF via activation of the MAP kinase cascade or via PI3 kinase dependent activation of protein kinase B.

Compounds that inhibit GSK-3 activity are useful in the treatment of diseases, disorders and conditions, wherein such an inhibition is beneficial eg in diseases, disorders and conditions related to GSK-3, in diseases, disorders and conditions related to a dysfunction of GSK-3, in diseases, disorders and conditions in which growth factor induced inhibition of GSK-3 is insufficient and in conditions in which glycogen synthase is insufficiently activated.

Type 1 diabetes, also known as insulin dependent diabetes mellitus (IDDM), is caused by an autoimmune destruction of insulin producing cells in the pancreas, leading to a lack of insulin. Thus, individuals with Type 1 diabetes require daily injections of the hormone to sustain life. Current methods of insulin administration, however, cannot reproduce the normal β cell's ability to precisely control blood glucose and other metabolic variables. Hence, the Type 1 diabetic remains susceptible to the long-term and devastating complications of diabetes, such as cardiovascular disease, retinopathy, nephropathy and neuropathy.

Type 2 diabetes, also known as non-insulin dependent diabetes mellitus (NIDDM), is the most common of all metabolic disorders and poses a major health problem worldwide. Type 2 diabetes results from defects in both insulin secretion and insulin action, but the exact underlying mechanism(s) causing the disease are not known. An elevation of hepatic glucose production contributes significantly to causing fasting hyperglycemia, whereas decreased insulin-mediated glucose uptake by muscle and fat is a major contributor to postprandial hyperglycemia. Moreover, the metabolic fate of glucose taken up by muscle is not normal in people with Type 2 diabetes. For example muscle glycogen synthase activity and glycogen synthesis have been shown to be impaired in Type 2 diabetes. The available treatments do not allow for a complete normalisation of the metabolic state and some of them are associated with side effects. The metabolic derangements created by hyperglycemia, together with the strong association between Type 2 diabetes, obesity, hypertension, and hyperlipidemia, lead to an extensive list of long-term complications, including a high rate of cardiovascular death due to accelerated atherosclerosis, as well as typical complications of diabetes such as retinopathy, nephropathy, and neuropathy.

Thus, there is still a need for novel approaches to treat diabetes.

Recently, it has been found that GSK-3 expression is elevated in muscle of people with Type 2 diabetes, and that the GSK-3 expression is inversely correlated with both glycogen synthase activity and glucose disposal. Thus, an increased GSK-3 expression may contribute to the impaired glycogen synthase activity and insulin resistance that occurs in Type 2 diabetes. Other recent experiments have suggested a role for GSK-3 in attenuating insulin action via its phosphorylation of insulin receptor substrate 1.

Recent studies using lithium salts also support the notion that inhibition of GSK-3 would be beneficial in the treatment of diabetes. It has long been known that lithium has a stimulatory effect on glucose metabolism, most prominently on glycogen synthesis. Treatment with lithium salts has also been shown to alleviate the diabetic state in both Type 1 and Type 2 diabetic patients. The molecular mechanism for these effects of lithium has until recently been unknown. However, it has now been found that lithium inhibits GSK-3. Although lithium might also have effects on other molecular targets than GSK-3, this finding contributes to explain the molecular effects of lithium, and supports that inhibition of GSK-3 leading to activation of glycogen synthase has significant effect on stimulation of glucose metabolism.

In conclusion, GSK-3 inhibitors may be useful for the treatment of metabolic disorders, such as IGT, Type 1 diabetes and Type 2 diabetes.

GSK-3 is also involved in biological pathways relating to Alzheimer's disease and GSK-3 inhibitors may be useful in the treatment thereof. Alzheimer's disease is characterized histopathologically by the presence of intraneuronal neurofibrillary tangles and the extracellular deposition of β amyloid in the brain, especially the hippocampus. The neurofibrillary tangles are made up of paired helical filaments (PHFs), the major protein subunit of which is the abnormally phosphorylated and glycosylated microtubule associated protein tau (τ). In the tangle bearing neurons in Alzheimer's disease, the normal cytoskeleton is disrupted and replaced with PHFs. GSK-3 is one of several kinases that phosphorylates tau in vitro on the abnormal sites characteristic of PHF-tau, and has also been demonstrated to do this in living cells. Furthermore, the GSK-3 inhibitor lithium blocks tau hyperphosphorylation in cells. Further evidence for a role of GSK-3 in Alzheimer's disease is provided by ia (i) the association of GSK-3 with presenellin 1, (ii) reduced cytotoxicity of β amyloid protein in neuronal cells incubated with GSK-3 antisense and (iii) 50% increased expression of GSK-3 in postsynaptic supernatants of Alzheimer's disease compared to normal brain tissue.

Lithium has been used for decades in the treatment of manic depression (bipolar disorder). The mechanism of action of lithium as a mood-stabilizing agent remains unknown, although effects on biological membranes and synaptic neurotransmission have been suggested. However, GSK-3 activity could be implicated in the etiology of bipolar disorder. One mechanism by which lithium and other GSK-3 inhibitors may act to treat bipolar disorder is to increase the survival of neurons subjected to aberrantly high levels of excitation induced by the neurotransmitter glutamate. Glutamate may also be implicated in mediating neurode generation following acute damage, eg in cerebral ischemia, traumatic brain injury and bacterial, viral, and prion infection. Excessive glutamate signalling has also been implicated in the chronic neuronal damage seen in diseases such as Huntington's chorea, Parkinson's disease, amyotrophic lateral sclerosis and multiple sclerosis. Consequently, GSK-3 inhibitors may be useful in the treatment of these and other neurodegenerative disorders. In connection with this it should be noted that lithium has a variety of biological effects that, if mediated through the inhibition of GSK-3, could provide an even broader application of GSK-3 inhibitors.

Furthermore, GSK-3 has been shown to phosphorylate the transcription factor NF-AT, which participates in the activation of early immune response genes. Phosphorylation prevents translocation of NF-AT to the nucleus, and thus blocks early immune responses. Thus, GSK-3 inhibitors may prolong and potentiate the immunostimulatory effect of certain cytokines, and such an effect could be beneficial in the use of cytokines for cancer or immunotherapy.

WO 98/16528 discloses purine derivates, which are stated to be effective as inhibitors of GSK-3. WO 99/65897 discloses pyrimidine and pyridine derivates, which are stated to be effective as inhibitors of GSK-3.

WO 99/21845 discloses 4-aminothiazole derivatives and their use as inhibitors of cyclindependent kinases (CDKs). These compounds are stated to be effective for the treatment of ia cancer. WO 00/75120 discloses diaminothiazoles and their use for inhibiting protein kinases. The compounds are stated to be useful for the treatment of disease conditions associated with tumor growth, cell proliferation or angiogenesis, such as cancer.

Furthermore, several references disclose the synthesis of 2,4-diaminothiazole derivates and/or the use thereof as reagents in different reactions and syntheses. See Binu, R et al., Synth. Commun. (1998), 28(19), 3625-3625; Binu, R. et al., Org. Prep. Proced. Int. (1998), 30(1), 93-96; Jenardanan, G. C. et al., Synth. Commun. (1997), 27(19), 3457-3462; Jenardanan, G. C. et al., Synth. Commun. (1997), 27(19), 3457-3462; Sauter, F. et al., Monatsh. Chem. (1997), 128(5), 503-508; Rajasekharan, K. N. et al., Synthesis (1986), (5), 353-5; Wobig, D., Liebigs Ann. Chem. (1984), (12), 1994-7; Frohlich, J. et al., Sci. Pharm. (1997), 65(3), 83-92; Gewald, K. et al., J. Prakt. Chem. (1967), 35(1-2), 97-104; and Gewald, K. etal., Monatsh. Chem. (1981), 112(12), 1393-404. However, these references neither disclose nor suggest any therapeutic use of the 2,4-diaminothiazole derivatives.

In view of the art's interest in GSK-3 inhibitors and the great potential thereof, the identification of potent and specific GSK-3 inhibitors would be a highly desirable contribution to the art. The present invention provides such a contribution to the art being based on the finding that the 2,4-diaminothiazole derivatives of the general formula (I) potently and specifically inhibit GSK-3.

The present compounds are accordingly useful in the treatment and/or prevention of a wide range of conditions and disorders in which an inhibition of GSK-3 is beneficial.

Furthermore, some of the compounds have been shown to potentiate the release of the glucose-induced stimulation of insulin secretion.

### DEFINITIONS

The following is a detailed definition of the terms used to describe the compounds of the invention.

“Halogen” designates an atom selected from the group consisting of F, Cl, Br and l.

The term “C₁₋₆-alkyl” in the present context designates a saturated, branched or straight hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl and the like.

The term “C₁₋₆-alkylene” in the present context designates a divalent saturated, branched or straight hydrocarbon group having from 1 to 6 carbon atoms. Representative examples include, but are not limited to, methylene, ethylene, propylene, butylene, pentylene, hexylene, and the like.

The term “C₁₋₆-alkoxy” in the present context designates a group —O—C₁₋₆-alkyl wherein C₁₋₆-alkyl is as defined above. Representative examples include, but are not limited to, methoxy, ethoxy, n-propoxy, isopropoxy, butoxy, isobutoxy, sec-butoxy, tert-butoxy, n-pentoxy, isopentoxy, neopentoxy, tert-pentoxy, n-hexoxy, isohexoxy and the like.

The term “C₁₋₆-alkanoyloxy” in the present context designates a group —OC(═O)H or —OC(═O)C₁₋₅-alkyl wherein C₁₋₅-alkyl designates a saturated, branched or straight hydrocarbon group having from 1 to 5 carbon atoms. Representative examples include, but are not limited to, formyloxy, acetoxy, n-propanoyloxy, isopropanoyloxy, butanoyloxy, isobutanoyloxy, secbutanoyloxy, tert-butanoyloxy, n-pentanoyloxy, isopentanoyloxy, neopentanoyloxy, tertpentanoyloxy, n-hexanoyloxy, isohexanoylxy and the like.

The term “C₁₋₆-alkylthio” in the present context designates a group —S—C₁₋₆-alkyl wherein C₁₋₆-alkyl is as defined above. Representative examples include, but are not limited to, methylthio, ethylthio, n-propylthio, isopropylthio, butylthio, isobutylthio, sec-butylthio, tertbutylthio, n-pentylthio, isopentylthio, neopentylthio, tert-pentylthio, n-hexylthio, isohexylthio and the like.

The term “C₂₋₆-alkenyl” as used herein represents a branched or straight hydrocarbon group having from 2 to 6 carbon atoms and at least one double bond. Examples of such groups include, but are not limited to, vinyl, 1-propenyl, 2-propenyl, isopropenyl, 1,3-butadienyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 2,4-hexadienyl, 5-hexenyl and the like.

The term “C₂₋₆-alkynyl” as used herein represents a branched or straight hydrocarbon group having from 2 to 6 carbon atoms and at least one triple bond. Examples of such groups include, but are not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 5-hexynyl, 2,4-hexadiynyl and the like.

The term “C₃₋₈-cycloalkyl” as used herein represents a saturated carbocyclic group having from 3 to 8 carbon atoms. Representative examples are cyclopropyl, cyclobutyl, cyclopentyl, cyclohexyl, cycloheptyl, cyclooctyl and the like.

The term “C₃₋₈-heterocyclyl” as used herein represents a saturated 3 to 8 membered ring containing one or more heteroatoms selected from nitrogen, oxygen and sulfur. Representative examples are pyrrolidinyl, piperidyl, piperazinyl, morpholinyl, thiomorpholinyl, aziridinyl, tetrahydrofuranyl and the like.

The term “aryl” as used herein represents a carbocyclic aromatic ring system such as phenyl, biphenyl, naphthyl, anthracenyl, phenanthrenyl, fluorenyl, indenyl, pentalenyl, azulenyl, biphenylenyl and the like. Aryl is also intended to include the partially hydrogenated derivatives of the carbocyclic aromatic systems enumerated above. Non-limiting examples of such partially hydrogenated derivatives are 1,2,3,4-tetrahydronaphthyl, 1,4-dihydronaphthyl and the like.

The term “heteroaryl” as used herein represents a heterocyclic aromatic ring system containing one or more heteroatoms selected from nitrogen, oxygen and sulfur such as furanyl, thiophenyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5- triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl, indazolyl, benzimidazolyl, benzthiazolyl, benzisothiazolyl, benzoxazolyi, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the heterocyclic systems enumerated above. Non-limiting examples of such partially hydrogenated derivatives are 2,3-dihydrobenzofuranyl, pyrrolinyl, pyrazolinyl, indolinyl, oxazolidinyl, oxazolinyl, oxazepinyl and the like.

“Aryl-C₁₋₆-alkyl”, “heteroaryl-C₁₋₆-alkyl” etc. means C₁₋₆-alkyl as defined above, substituted by an aryl or heteroaryl as defined above, for example:

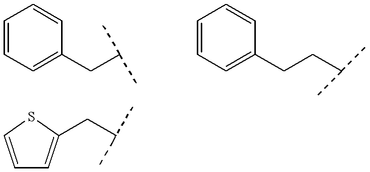

Certain of the above defined terms may occur more than once in the structural formulae, and upon such occurrence each term shall be defined independently of the other.

The term “GSK-3” as used herein is intended to mean GSK-3α and/or GSK-α.

### DETAILED DESCRIPTION OF THE INVENTION

Accordingly, the present invention relates to the use of a compound of the formula (I):

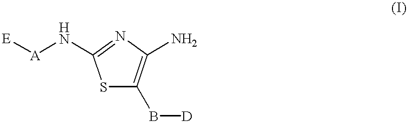

wherein

E is C₁₋₆-alkyl, C₂₋₆-alkenyl, C₂₋₆-alkynyl, C₁₋₆-alkylthio, C₁₋₆-alkoxy, C₁₋₆-alkanoyloxy, —C(═O)OH, —C(═O)O—C₁₋₆-alkyl, or

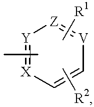

X, Y, Z and V independently are ═CH— or ═N—, with the proviso that at least two of X, Y, Z and V are ═CH—,

R¹ and R² which may be the same or different independently are selected from

hydrogen, hydroxy, halogen, cyano, nitro, —NR³R⁴, —C(═O)NR³R⁴, —OC(═O)NR³R⁴, —OCH₂C(═O)NR³R⁴, C₁₋₆-alkoxy, —C(═O)OR³, —C(═O)R³, —NHC(═O)R³, —CHF₂, —CF₃, —OCF₃—-OCHF₂, —OCH₂CF₃, —OCF₂CHF₂, —SCF₃, —SR³, —S(═O)R³, —S(═O)₂R³, —S(═O)₂NH₂,

wherein R³ and R⁴ which may be the same or different independently are selected from hydrogen and C₁₋₆-alkyl or R³ and R⁴ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR³R⁶, —C(═)NR⁵R⁶,—OC(═O)NR⁵R⁶, —OCH₂C(═O)NR⁵R⁶, C₁₋₆-alkoxy, —C(═O)OR⁵, —C(═O)R⁵, —NHC(═O)R⁵, —CHF₂, —CF₃, —OCF₃, —OCF₂, —OCH₂CF₃, —OCF₂CHF₂, —SCF₃, —SR⁵, —S(═O)R⁵, —S(═O)₂R⁵, —S(═O)₂NH₂, wherein R⁵ and R⁶ which may be the same or different independently are selected from hydrogen and C₁₋₆-alkyl or R⁵ and R⁶ together with the nitrogen atom to which they are attached form a 3 to 8 membered cyclic ring optionally containing one or two further heteroatoms selected from oxygen, sulfur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkyl-carbonyl, heteroaroyl, C₃₋₈-heterocyclylcarbonyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, which may optionally be substituted with one to three substituents selected from

hydroxy, halogen, cyano, nitro, —NR⁷R⁸, —C(═O)NR⁷R⁸, —OC(═O)NR⁷R⁸, —OCH₂C(═O)NR⁷R⁸, C₁₋₆-alkoxy, —C(═O)OR⁷, —C(═O)R⁷, —NHC(═O)R⁷, —CHF₂, —CF₃, —OCF₃, —OCHF₂, —OCH₂CF₃, —OCF₂CHF₂, —SCF₃, —SR⁷, —S(═O)R⁷, —S(═O)₂R⁷, —S(═O)₂NH₂,

wherein R⁷ and R⁸ which may be the same or different independently are selected from hydrogen and C₁₋₆-alkyl or R⁷ and R⁸ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR⁹R¹⁰, —C(═O)NR⁹R¹⁰, —OC(═O)NR⁹R¹⁰, —OCH₂C(═O)NR⁹R¹⁰, C₁₋₆-alkoxy, —C(═O)OR⁹, —C(═O)R⁹, —NHC(═O)R⁹, —CHF₂, —CF₃, —OCF₃, —OCHF₂, —OCH₂CF₃, —OCF₂CHF₂, —SCF₃, —SR⁹, —S(═O)R⁹, —S(═O)₂R⁹, —S(═O)₂NH₂,

wherein R⁹ and R¹⁰ which may be the same or different independently are selected from hydrogen and C₁₋-alkyl or R⁹ and R¹⁰ together with the nitrogen atom to which they are attached form a 3 to 8 membered cyclic ring optionally containing one or two further heteroatoms selected from oxygen, sulfur and nitrogen,

A is a valence bond, C₁₋₆-alkylene or —C(═O)—,

B is a valence bond, —C(═O)—, —S(═O)—, —S(═O)₂— or —C(═N-OR¹¹)—,

R¹¹ is hydrogen, C₁₋₆-alkyl or aryl-C₁₋₆-alkyl,

D is

hydroxy, halogen, cyano, nitro, —NR¹²R¹³, —N(R¹²)OR¹³, —C(═O)NR¹²R¹³, —OC(═O)NR¹²R¹³, —OCH₂C(═O)NR¹²R¹³, C₁₋₆-alkoxy, —C(═O)OR¹², —C(═O)R¹², —NHC(═O)R¹², —CHF₂, —CF₃ —OCF₃, —OCHF₂, —OCH₂CF₃, —OCF₂CHF₂, —SCF₃, —SR¹², —S(═O)R¹², —S(═O)₂R¹², —S(═O)₂NH₂,

wherein R¹² and R¹³ which may be the same or different independently are selected from hydrogen and C₁₋₆-alkyl or R¹² and R¹³ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR ⁴R¹⁵, —C(═O)NR¹⁴R¹⁵, —OC(═O)NR¹⁴R¹⁵, —OCH₂C(═O)NR¹⁴R¹⁵, C₁₋₆-alkoxy, —C(═O)OR¹⁴, —C(═O)R¹⁴, —NHC(═O)R¹⁴, —CHF₂, —CF₃, —OCF₃, —OCHF₂, —OCH₂CF₃, —OCF₂CHF₂, —SCF₃, —SR¹⁴, —S(═O)R¹⁴, —S(═O)₂R¹⁴, —S(═O)₂NH₂,

wherein R¹⁴ and R¹⁵ which may be the same or different independently are selected from hydrogen and C₁₋₆-alkyl or R¹⁴ and R¹⁵ together with the nitrogen atom to which they are attached form a 3 to 8 membered cyclic ring optionally containing one or two further heteroatoms selected from oxygen, sulfur and nitrogen,

aryl, C₃₋₈-cycloalkyl, heteroaryl, C₃₋₈-heterocyclyl, aryl-C₁,-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkyl-carbonyl, heteroaroyl, C₃₋₈-heterocyclylcarbonyl, —O-aryl, —O-C₃₋₈-cycloalkyl, —O-heteroaryl, —O—C₃₋₈-heterocyclyl, —S-aryl, —S-C₃₋₈-cycloalkyl, —S-heteroaryl, —S—C₃₋₈-heterocyclyl, —NH-aryl, —NH-heteroaryl,

which may optionally be substituted with one to three substituents selected from

hydroxy, halogen, cyano, nitro, —NR¹⁶R¹⁷, —C(═O)NR¹⁶R¹⁷, —OC(═O)NR¹⁶R¹⁷, —OCH₂C(═O)NR¹⁶R¹⁷, C₁₋₆-alkoxy, —C(═O)OR¹⁶, —C(═O)R¹⁶, —NHC(═O)R¹⁶, —CHF₂, —CF₃, —OCF₃, —OCHF₂, —OCH₂CF₃, —OCF₂CHF₂, —SCF₃, —SR¹⁶, —S(═O)R¹⁶, —S(═O)₂R¹⁶, —S(═O)₂NH₂,

wherein R¹⁶ and R¹⁷ which may be the same or different independently are selected from hydrogen and C₁₋₆-alkyl or R¹⁶ and R¹⁷ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR¹⁸R¹⁹, —C(═O)NR¹⁸R¹⁹, —OC(═O)NR¹⁸R¹⁹, —OCH₂C(═O)NR¹⁸R¹⁹, C₁₋₆-alkoxy, —C(═O)OR¹⁸, —C(═O)R¹⁸, —NHC(═O)R¹⁸, —CHF₂, —CF₃, —OCF₃, —OCHF₂, —OCH₂CF₃, —OCF₂CHF₂, —SCF₃, —SR¹⁸, —S(═O)R¹⁸, —S(═O)₂R¹⁸, —S(═O)₂NH₂,

wherein R¹⁸ and R¹⁹ which may be the same or different independently are selected from hydrogen and C₁₋₆-alkyl or R¹⁸ and R¹⁹ together with the nitrogen atom to which they are attached form a 3 to 8 membered cyclic ring optionally containing one or two further heteroatoms selected from oxygen, sulfur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C1-6-alkyl, aryl-C₁₋₆-alkoxy, C₃₋₈-cycloalkyl-C₁₋₆-alkoxy, heteroaryI-C₁₋₆-alkoxy, C₃₋₈-heterocyclyl-C₁₋₆-alkoxy, aroyl, C₃₋₈-cycloalkyl-carbonyl, heteroaroyl, C₃₋₈-heterocyclylcarbonyl, —O-aryl, —O-C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,

which may optionally be substituted with one to three substituents selected from hydroxy, halogen, cyano, nitro, —NR²⁰R²¹, —C(═O)NR²⁰R²¹, —OC(═O)NR²⁰R²¹, —OCH₂C(═O)NR²OR²¹, C₁₋₆-alkyl, C₂₋₆-alkenyl, C₂₋₆-alkynyl, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,

wherein R²⁰ and R²¹ which may be the same or different independently are selected from hydrogen and C₁₋₆-alkyl or R²⁰ and R²¹ together with the nitrogen atom to which they are attached form a 3 to 8 membered cyclic ring optionally containing one or two further heteroatoms selected from oxygen, sulfur and nitrogen,

as well as any optical or geometric isomer or tautomeric form thereof including mixtures of these or a pharmaceutically acceptable salt thereof for the preparation of a pharmaceutical composition for the treatment and/or prevention of diseases and disorders wherein an inhibition of GSK-3 is beneficial.

In another aspect the present invention relates to the use of a compound of the general formula (I′):

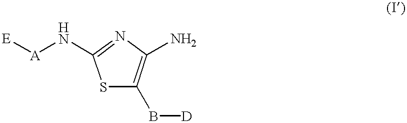

wherein

E is C₁₋₆-alkyl or

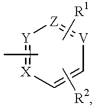

X, Y, Z and V independently are ═CH— or ═N—, with the proviso that at least two of X, Y, Z and V are ═CH—,

R¹ and R² which may be the same or different independently are selected from

hydrogen, hydroxy, halogen, cyano, nitro, —NR³R⁴, —C(═O)NR³R⁴, —OC(═O)NR³R⁴, —OCH₂C(═O)NR³R⁴, C₁₋₆-alkoxy, —C(═O)OR³, —C(═O)R³, —NHC(═O)R³, —CHF₂, —CF₃, —OCF₃, —OCHF₂, —OCH₂CF₃, —OCF₂CHF₂, —SCF₃, —SR³, —S(═O)R³, —S(═O)₂R³, —S(═O)₂NH₂,

wherein R³ and R⁴ which may be the same or different independently are selected from hydrogen and C₁₋₆-alkyl or R³ and R⁴ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-,alkenyl, C₂₋₆-alkynyl,

which may optionally be substituted with one or two substituents selected from hydroxy, halogen, cyano, nitro, —NR⁵R⁶, —C(═O)NR⁵R⁶, —OC(═O)NR⁵R⁶, —OCH₂C(═O)NR⁵R⁶, C₁₋₆-alkoxy, —C(═O)OR⁵, —C()R⁵, —NHC(═O)R⁵, —CHF₂, —CF₃, —OCF₃, —OCHF₂, —OCH₂CF₃, —OCF₂CHF₂, —SCF₃, —SR⁵, —S(═O)R⁵, —S(═O)₂R⁵, —S(═O)₂NH₂,

wherein R⁵ and R⁶ which may be the same or different independently are selected from hydrogen and C₁₋₆-alkyl or R⁵ and R⁶ together with the nitrogen atom to which they are attached form a 3 to 8 membered cyclic ring optionally containing one or two further heteroatoms selected from oxygen, sulfur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkyl-carbonyl, heteroaroyl, C₃₋₈-heterocyclylcarbonyl, —O-aryl, —O-C₃₋₈-cycloalkyl, —O-heteroaryl, —O—C₃₋₈-heterocyclyl, —S-aryl, —S—C₃₋₈-cycloalkyl, —S-heteroaryl, —S-C₃₋₈-heterocyclyl,

which may optionally be substituted with one to three substituents selected from

hydroxy, halogen, cyano, nitro, —NR⁷R⁸, —C(═O)NR⁷R⁸, —RC(═O)NR⁷R⁸, —OCH₂C(═O)NR⁷R⁸, C₁₋₆-alkoxy, —C(═O)OR⁷, —C(═O)R⁷, —NHC(═O)R⁷, —CHF₂, —CF₃, —OCF₃, —OCHF₂, —OCH₂CF₃, —OCF₂CHF₂, —SCF₃, —SR⁷, —S(═O)R⁷, —S(═O)₂R⁷, —S(═O)₂N H₂,

wherein R⁷ and R⁸ which may be the same or different independently are selected from hydrogen and C₁₋₆-alkyl or R⁷ and R⁸ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR⁹R¹⁰, —C(═O)NR⁹R¹⁰, —OC(═O)NR⁹R¹⁰, —OCH₂C(═O)NR⁹R¹⁰, C₁₋₆-alkoxy, —C(═O)OR⁹, —C(═O)R⁹, —NHC(═O)R⁹, —CHF₂, —CF₃, —OCF₃, —OCHF₂, —OCH₂CF₃, —OCF₂CHF₂, —SCF₃, —SR⁹, —S(═O)R⁹, —S(═O)₂R⁹, —S(═0)₂NH₂,

wherein R⁹ and R¹⁰ which may be the same or different independently are selected from hydrogen and C₁₋₆-alkyl or R⁹ and R¹⁰ together with the nitrogen atom to which they are attached form a 3 to 8 membered cyclic ring optionally containing one or two further heteroatoms selected from oxygen, sulfur and nitrogen,

A is a valence bond, C₁₋₆-alkylene or —C(═O)—,

B is a valence bond, —C(═O)—, —S(═O)—, —S(═O)₂— or —C(═N—OR¹¹)—,

R¹¹ is hydrogen, C₁₋₆-alkyl or aryl-C₁₋₆-alkyl,

D is

hydroxy, halogen, cyano, nitro, —NR ²R¹³, —C(═O)NR¹²R¹³, —OC(═O)NR¹²R¹³, —OCH₂C(═O)NR ¹²R¹³, C₁₋₆-alkoxy, —C(═O)OR¹², —C(═O)R¹², —NHC(═O)R¹², —CHF₂, —CF₃, —OCF₃, —OCHF₂, —OCH₂CF₃, —OCF₂CHF₂, —SCF₃, —SR¹², —S(═O)R¹², —S(═O)₂R¹², —S(═O)₂NH₂,

wherein R¹² and R¹³ which may be the same or different independently are selected from hydrogen and C₁₋₆-alkyl or R¹² and R¹³ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR¹⁴R¹⁵, —C(═O)NR¹⁴R¹⁵, —OC(═O)NR¹⁴R¹⁵, —OCH₂C(═O)NR¹⁴R¹⁵, C₁₋₆-alkoxy, —C(═O)OR¹⁴, —C(═O)R¹⁴, —NHC(═O)R¹⁴, —CHF₂, —CF₃, —OCF₃, —OCHF₂, —OCH₂CF₃, —OCF₂CHF₂, —SCF₃, —SR¹⁴, —S(═O)R¹⁴, —S(═0)₂R¹⁴, —S(═O)₂NH₂,

wherein R¹⁴ and R¹⁵ which may be the same or different independently are selected from hydrogen and C₁₋₆-alkyl or R¹⁴ and R¹⁵ together with the nitrogen atom to which they are attached form a 3 to 8 membered cyclic ring optionally containing one or two further heteroatoms selected from oxygen, sulfur and nitrogen,

aryl, C₃₋₈-cycloalkyl, heteroaryl, C₃₋₈-heterocyclyl, aryl-C₁₋₆-alkyl, C3-8-cycloalkyl-C₁₋₆alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkyl-carbonyl, heteroaroyl, C₃₋₈-heterocyclylcarbonyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,

which may optionally be substituted with one to three substituents selected from

hydroxy, halogen, cyano, nitro, —NR¹⁵R¹⁷, —C(═O)NR¹⁶R¹⁷, —OC(═O)NR¹⁶R¹⁷ —OCH₂C(═O)NR¹⁶R¹⁷, C₁₋₆-alkoxy, —C(═O)OR¹⁶, —C(═O)R¹⁶, —NHC(═O)R¹⁶, —CHF₂, —CF₃, —OCF₃, —OCHF₂, —OCH₂CF₃, —OCF₂CHF₂, —SCF₃, —SR¹⁶, —S(═O)R¹⁶, —S(═O)₂R¹⁶, —S(═O)₂NH₂,

wherein R¹⁶ and R¹⁷ which may be the same or different independently are selected from hydrogen and C₁₋₆-alkyl or R¹⁶ and R¹⁷ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR¹⁸R¹⁹, —C(═O)NR¹⁸R¹⁹, —OC(═O)NR¹⁸R¹⁹, —OCH₂C(═O)NR¹⁸R¹⁹, C₁₋₆-alkoxy, —C(═O)OR¹⁸, —C(═O)R¹⁸, —NHC(═O)R¹⁸, —CHF₂, —CF₃, —OCF₃, —OCHF₂, —OCH₂CF₃, —OCF₂CHF₂, —SCF₃, —SR¹⁸, —S(═O)R¹⁸, —S(═O)₂R¹⁸, —S(═O)₂NH₂,

wherein R¹⁸ and R¹⁹ which may be the same or different independently are selected from hydrogen and C₁₋₆-alkyl or R¹⁸ and R¹⁹ together with the nitrogen atom to which they are attached form a 3 to 8 membered cyclic ring optionally containing one or two further heteroatoms selected from oxygen, sulfur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkylcarbonyl, heteroaroyl, C₃₋₈-heterocyclylcarbonyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,

which may optionally be substituted with one to three substituents selected from hydroxy, halogen, cyano, nitro, —NR²⁰R²¹, —C(═O)NR²⁰R²¹, —OC(═O)NR²⁰R²¹, —OCH₂C(═O) NR²⁰R²¹, C₁₋₆-alkyl, C₂₋₆-alkenyl, C₂₋₆-alkynyl, C₁₋₆-alkoxy, —C(═O)OR²⁰, —C(═O)R²⁰, —NHC(═O)R²⁰, —CHF₂, —CF₃, —OGF₃, —OCHF₂, —OCH₂CF₃, —OCF₂CHF₂, —SCF₃, —SR²⁰, —S(═O)R²⁰, —S(═O)₂R²⁰, —S(═O)₂NH₂,

wherein R²⁰ and R²¹ which may be the same or different independently are selected from hydrogen and C₁₋₆-alkyl or R²⁰ and R²¹ together with the nitrogen atom to which they are attached form a 3 to 8 membered cyclic ring optionally containing one or two further heteroatoms selected from oxygen, sulfur and nitrogen,

as well as any optical or geometric isomer or tautomeric form thereof including mixtures of these or a pharmaceutically acceptable salt thereof for the preparation of a pharmaceutical composition for the treatment and/or prevention of diseases and disorders wherein an inhibition of GSK-3 is beneficial.

It should be understood that R¹, R² and A may be attached to any one of the ring atoms including X, Y, Z and V whenever they are different from ═N—.

Preferably, A is a valence bond or C₁₋₆-alkylene.

Preferably, E is C₁₋₆-alkyl, C₂₋₆-alkoxy, —C(O)O—C₁₋₆-alkyl or

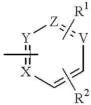

wherein X, Y, Z, V, R¹ and R² are as defined for formula (I).

Preferably, X, Y, Z and V are all ═CH—.

Preferably, R¹ and R² which may be the same or different are independently selected from

hydrogen, hydroxy, halogen, cyano, nitro, —NR³R⁴, —C(═O)NR³R⁴, —OC(═O)NR³R⁴, —OCH₂C(═O)NR³R⁴, C₁₋₆-alkoxy, —C(═O)OR³, —C(═O)R³, —NHC(═O)R³, —CHF₂, —CF₃, —OCF₃, —OCHF₂, —OCH₂CF₃, —OCF₂CHF₂, —SCF₃, —SR³, —S(═O)R³, —S(═O)₂R³, —S(═O)₂NH₂,

wherein R³ and R⁴ which may be the same or different independently are selected from hydrogen and C₁₋₆-alkyl or R³ and R⁴ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR⁵R⁶, —C(═O)NR⁵R⁶, —OC(═O)NR⁵R⁶, —OCH₂C(═O)NR⁵R⁶, C₁₋₆-alkoxy, —C(═O)OR⁵, —C(═O)R⁵, —NHC(═O)R⁵, —CHF₂, —CF₃, —OCF₃, —OCHF₂, —OCH₂CF₃, —OCF₂CHF₂, —SCF₃, —SR⁵, —S(═O)R⁵, —S(═O)₂R⁵, —S(═O)₂NH₂,

wherein R⁵ and R⁶ which may be the same or different independently are selected from hydrogen and C₁₋₆-alkyl or R⁵ and R⁶ together with the nitrogen atom to which they are attached form a 3 to 8 membered cyclic ring optionally containing one or two further heteroatoms selected from oxygen, sulfur and nitrogen.

More preferably, R¹ and R² which may be the same or different are independently selected from hydrogen or halogen.

Even more preferably, R¹ and R² are both hydrogen.

Preferably, B is —C(═O)— or a valence bond.

In a preferred embodiment of the invention, D is

hydroxy, —NR¹²R¹³, —C(O)R¹², cyano, —N(R¹²)OR¹³, C₁₋₆-alkoxy, —C(═O)OR¹², —CF₃, C₁₋₆-alkyl,

wherein R¹² and R¹³ which may be the same or different independently are selected from hydrogen and C₁₋₆-alkyl or R¹² and R¹³ together with the nitrogen atom to which they are attached form a 3 to 8 membered cyclic ring optionally containing one or two further heteroatoms selected from oxygen, sulfur and nitrogen,

aryl, C₃₋₈-cycloalkyl, heteroaryl, C₃₋₈-heterocyclyl, aryl-C₁₋₆-alkoxy, —NH-aryl,

which may optionally be substituted with one to three substituents selected from

hydroxy, halogen, cyano, nitro, —NR¹⁶R¹⁷, —C(═O)NR¹⁶R¹⁷, —OC(═O)NR¹⁶R¹⁷, —OCH₂C(═O)NR¹⁶R¹⁷, C₁₋₆-alkoxy, —C(═O)OR¹⁶, —C(═O)R¹⁶, —NHC(═O)R¹⁶, —CHF₂, —CF₃, —OCF₃, —OCHF₂, —OCH₂CF₃, —OCF₂CHF₂, —SCF₃, —SR¹⁶, —S(═O)R¹⁶, —S(═O)₂R¹⁶, —S(═O)₂NH₂,

wherein R¹⁶ and R¹⁷ which may be the same or different independently are selected from hydrogen and C₁₋₆-alkyl or R¹⁶ and R¹⁷ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR¹⁸R¹⁹, —C(═O)NR¹⁸R¹⁹, —OC(═O)NR¹⁸R¹⁹, —OCH₂C(═O)NR¹⁸R¹⁹, C₁₋₆-alkoxy, —C(═O)OR¹⁸, —C(═O)R¹⁸, —NHC(═O)R¹⁸, —CHF₂, —CF₃, —OCF₃, —OCHF₂, —OCH₂CF₃, —OCF₂CHF₂, —SCF₃, —SR¹⁸, —S(═O)R¹⁸, —S(═O)₂R¹⁸, —S(═O)₂NH₂,

wherein R¹⁵ and R¹⁹ which may be the same or different independently are selected from hydrogen and C₁₋₆-alkyl or R¹⁸ and R¹⁹ together with the nitrogen atom to which they are attached form a 3 to 8 membered cyclic ring optionally containing one or two further heteroatoms selected from oxygen, sulfur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkyl-carbonyl, heteroaroyl, C₃₋₈-heterocyclylcarbonyl, —O-aryl, —O—C₃₋₈-cycloalkyl, —O—heteroaryl, —O—C₃₋₈-heterocyclyI, —S-aryl, —S—C₃₋₈-cycloalkyl, —S-heteroaryl, —S—C₃₋₈-heterocyclyl,

which may optionally be substituted with one to three substituents selected from hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆-alkyl, C₂₋₆-alkenyl, C₂₋₆-alkynyl, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,

wherein R²⁰ and R²¹ which may be the same or different independently are selected from hydrogen and C₁₋₆-alkyl or R²⁰ and R²¹ together with the nitrogen atom to which they are attached form a 3 to 8 membered cyclic ring optionally containing one or two further heteroatoms selected from oxygen, sulfur and nitrogen.

In another preferred embodiment of the invention D is C₃₋₈-cycloalkyl, aryl or heteroaryl, which are optionally substituted with one or more substituents selected from halogen, C₁₋₆-alkyl and phenyl-C₁₋₆-alkoxy.

In yet another preferred embodiment of the invention D is

hydroxy, —NR¹²R¹³, C₁₋₆-alkoxy, —C(═O)OR¹², —CF₃, C₁₋₆-alkyl,

wherein R¹² and R¹³ which may be the same or different independently are selected from hydrogen and C₁₋₆-alkyl or R¹² and R¹³ together with the nitrogen atom to which they are attached form a 3 to 8 membered cyclic ring optionally containing one or two further heteroatoms selected from oxygen, sulfur and nitrogen,

aryl, C₃₋₈-cycloalkyl, heteroaryl, C₃₋₈-heterocyclyl, aryl-C₁₋₆-alkoxy,

which may optionally be substituted with one to three substituents selected from

hydroxy, halogen, cyano, nitro, —NR¹⁶R¹⁷, —C(═O)NR¹⁶R¹⁷, —OC(═O)NR¹⁶R¹⁷, —OCH₂C(═O)NR¹⁶R¹⁷, C₁₋₆-alkoxy, —C(═O)OR¹⁶, —C(═O)R¹⁶, —NHC(═O)R¹⁶, —CHF₂, —CF₃, —OCF₃, —OCHF₂, —OCH₂CF₃, —OCF₂CHF₂, —SCF₃, —SR¹⁶, —S(═O)R¹⁶, —S(═O)₂R¹⁶, —S(═O)₂NH₂,

wherein R¹⁶ and R¹⁷ which may be the same or different independently are selected from hydrogen and C₁₋₆-alkyl or R¹⁶ and R¹⁷ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR¹⁸R¹⁹, —C(═O)NR¹⁸R¹⁹, —OC(═O)NR¹⁸R¹⁹, —OCH₂C(═O)NR¹⁸R¹⁹, C₁₋₆-alkoxy, —C(═O)OR¹⁸, —C(═O)R¹⁸, —NHC(═O)R¹⁸, —CHF₂, —CF₃, —OCF₃, —OCHF₂, —OCH₂CF₃, —OCF₂CHF₂, —SCF₃, —SR¹⁸, —S(═O)R¹⁸, —S(═O)₂R¹⁸, —S(═O)₂NH₂,

wherein R¹⁸ and R¹⁹ which may be the same or different independently are selected from hydrogen and C₁₋₆-alkyl or R¹⁸ and R¹⁹ together with the nitrogen atom to which they are attached form a 3 to 8 membered cyclic ring optionally containing one or two further heteroatoms selected from oxygen, sulfur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cyclo-alkyl-C₁₋₆-alkoxy, heteroaryl-C₁₋₆-alkoxy, C₃₋₈-heterocyclyl-C₁₋₆-alkoxy, aroyl, C₃₋₈-cycloalkylcarbonyl, heteroaroyl, C₃₋₈-heterocyclylcarbonyl, —O-aryl, —O—C₃₋₈-cyclo-alkyl, —O-heteroaryl, —O—C₃₋₈-heterocyclyl, —S-aryl, —S—C₃₋₈-cycloalkyl, —S-heteroaryl, —S—C₃₋₈-heterocyclyl,

which may optionally be substituted with one to three substituents selected from hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆-alkyl, C₂₋₆-alkenyl, C₂₋₆-alkynyl, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,

wherein R²⁰ and R²¹ which may be the same or different independently are selected from hydrogen and C₁₋₆-alkyl or R²⁰ and R²¹ together with the nitrogen atom to which they are attached form a 3 to 8 membered cyclic ring optionally containing one or two further heteroatoms selected from oxygen, sulfur and nitrogen.

In yet a preferred embodiment of the invention D is

hydroxy, —NR¹²R¹³, C₁₋₆-alkoxy, —C(═O)OR¹², —CF₃, C₁₋₆-alkyl,

wherein R¹² and R¹³ which may be the same or different independently are selected from hydrogen and C₁₋₆-alkyl,

cyclopropyl, phenyl, naphthyl, morpholino, pyridinyl, tetrahydropyridinyl, thiophenyl, benzothiophenyl, phenyl-C₁₋₆-alkoxy,

which may optionally be substituted with one to three substituents selected from

hydroxy, halogen, nitro, —NR¹⁶R¹⁷, C₁₋₆-alkoxy, —CF₃, —OCF₃, C₁₋₆-alkyl,

wherein R¹⁶ and R¹⁷ which may be the same or different independently are selected from hydrogen and C₁₋₆-alkyl,

phenyl, phenyl-C₁₋₆-alkoxy,

which may optionally be substituted with one to three substituents selected from halogen, nitro, —NR²⁰R²¹, C₁₋₆-alkoxy, —CF₃, —OCF₃, C₁₋₆-alkyl,

wherein R²⁰ and R²¹ which may be the same or different independently are selected from hydrogen and C₁₋₆-alkyl.

In still another preferred embodiment of the invention D is

hydroxy, —NR¹²R¹³, C₁₋₆-alkoxy, —C(═O)OR¹², —CF₃, C₁₋₆-alkyl,

wherein R¹² and R¹³ which may be the same or different independently are selected from hydrogen and C₁₋₆-alkyl, cyclopropyl, phenyl, naphthyl, morpholino, pyridinyl, thiophenyl, benzothiophenyl, phenyl-C₁₋₆-alkoxy,

which may optionally be substituted with one to three substituents selected from

hydroxy, halogen, nitro, —NR¹⁶R¹⁷, C₁₋₆-alkoxy, —CF₃, —OCF₃, C₁₋₆-alkyl,

wherein R¹⁶ and R¹⁷ which may be the same or different independently are selected from hydrogen and C₁₋₆-alkyl,

phenyl, phenyl-C₁₋₆-alkoxy,

which may optionally be substituted with one to three substituents selected from halogen, nitro, —NR²⁰R²¹, C₁₋₆-alkoxy, —CF₃, —OCF₃, C₁₋₆-alkyl,

wherein R²⁰ and R²¹ which may be the same or different independently are selected from hydrogen and C₁₋₆-alkyl.

More preferably, D is

cyclopropyl, phenyl, pyridinyl, tetrahydropyridinyl, thiophenyl,

which may optionally be substituted with one to three substituents selected from

hydroxy, halogen, nitro, —NR¹⁶R¹⁷, C₁₋₆-alkoxy, —CF₃, —OCF₃, C₁₋₆-alkyl,

wherein R¹⁶ and R¹⁷ which may be the same or different independently are selected from hydrogen and C₁₋₆-alkyl,

phenyl, phenyl-C₁₋₆-alkoxy,

which may optionally be substituted with one to three substituents selected from halogen, nitro, —NR²⁰R²¹, C₁₋₆-alkoxy, —CF₃, —OCF₃, C₁₋₆-alkyl,

wherein R²⁰ and R²¹ which may be the same or different independently are selected from hydrogen and C₁₋₆-alkyl.

Even more preferably, D is cyclopropyl, phenyl, pyridinyl, tetrahydropyridinyl or thiophenyl, which are optionally substituted with one to three substituents selected from halogen, C₁₋₆-alkyl and phenyl-C₁₋₆-alkoxy, such as cyclopropyl or pyridinyl.

In a preferred embodiment of the invention the compound is selected from

(4-amino-2-phenylaminothiazol-5-yl)cyclopropylmethanone,

(4-amino-2-phenylaminothiazol-5-yl)-(4-fluorophenyl)methanone,

(4-amino-2-phenylaminothiazol-5-yl)-(4-chlorophenyl)methanone,

(4-amino-2-phenylaminothiazol-5-yl)phenylmethanone,

(4-amino-2-phenylaminothiazol-5-yl)-(3-nitrophenyl)methanone,

(4-amino-2-phenylaminothiazol-5-yl)-(4-nitrophenyl)methanone,

[4-amino-2-(4-chloro-phenylamino )thiazol-5-yl]phenylmethanone,

(4-amino-2-(ethylaminothtiazol-5-yl)phenylmethanone,

(4-amino-2-phenylaminothiazol-5-yl)phendiethyl methanone,

(4-amino-2-phenylaminothiazol-5-yl)pyridin-3-ylmethoyhnlmtanone,

4-amino-2-phenylaminothiazole-5-carboxylic acid tert-butyl ester,

1-(amino-2-phenylaminothiazol-5-yl)-2,2-dimethylpropan-1-one,

1-(4-amino-2-phenylaminothiazol-5-yl)propan-1-one,

(4-amino-2-phenylaminothiazol-5-yl)-(3,4-difluoro phenyl)methanone,

(4-amino-2-phenylaminothiazol-5-yl)-(3-fluorophenyl)methanone,

(4-amino-2-phenylaminothiazol-5-yl)naphtha len-2-ylmethanone,

(4-amino-2-phenylaminothiazol-5-yl)biphenyl-4-ylmethanone,

(4-amino-2-phenylaminothiazol-5-yl)-(3-benzylo xyphenyl)methanone,

[4-amino-2-(4-bromophenylamino)-thiazol-5-yl]cyclopropylmethanone,

N-(4-amino-5-cyclopropanecarbonylthiazol-2-yl)benzamide,

(4-amino-2-phenylaminothiazol-5-yl)-(3,4-dichlorophenyl)methanone,

(4-amino-2-phenylaminothiazol-5-yl)-(3-methylbenzyo[b]thiophen-2-yl)methanone,

(4-amino-2-phenylaminothiazol-5-yl)-(5-chlorothiophen-2-yl)methanone,

(4-amino-2-phenylaminothiazol-5-yl)-(2-methoxyphenyl)methanone,

(4-amino-2-phenylaminothiazol-5-yl)-(3-methoxyphenyl)methanone,

(4-amino-2-phenylaminothiazol-5-yl)-(4-methoxyphenyl)methanone,

(4-amino-2-phenylaminothiazol-5-yl)-(2-nitrophenyl)methanone,

(4-amino-2-phenylaminothiazol-5-yi)-(4-chloro-3-methylphenylemethanone,

(4-amino-2-phenylaminothiazol-5-yl)-(3,4-dihydroxyphenyl)methanone,

(4-amino-2-phenylaminothiazol-5-yl)-(2,4-dimethoxyphenyl)methanone,

[4-amino-2-(4-bromo-phenylamino)thiazol-5-yl]pyridin-3-yl-methanone,

(4-amino-2-propylaminothiazol-5-yl)pyridin-3-yl-methanone,

(4-amino-2-phenylaminothiazol-5-yl)pyridin-2-yl-methanone,

(4-amino-2-phenylaminothiazol-5-y)-pyridin-4-yl-methanone,

(4-amino-2-phenylaminothiazol-5-yl)-(2,4,6-trimethylphenyl)methanone,

(4-amino-2-phenylaminothiazol-5-yl)thiophen-2-yl-methanone,

(4-amino-2-phenylaminothiazol-5-yl)-(2,6-dimethoxyphenyl)methanone,

4-amino-2-phenylaminothiazole-5-carboxylic acid methyl ester,

(4-amino-2-phenylaminothiazole-5-yl)thiophen-3ylmethanone,

(4-amino-2-phenylaminothiazol-5-yl)-benzothiophen-3-yl-methanone,

(4-amino-2-phenylaminothiazol-5-yl)-(2,6-difuorophenyl)methanone,

(4-amino-2-phenylaminothiazol-5-yl)-(2,6-dichlorophenyl)methanone,

4-amino-2phenylaminothiazole-5-carbonitrile,

1-(4-amino-2-phenylaminothiazol-5-yl)ethanone,

4-amino-2-phenylaminothiazole-5-carboxylic acid phenylamide,

4-amino-2-phenylaminothiazole-5-carboxylic acid methoxymethylamide,

[4-amino-2-(pyridin-3-ylamino)thiazol-5-yl]phenylmethanone,

4-amino-2-phenylaminothiazole-5-carboxylic acid amide,

(4-amino-2-phenylaminothiazol-5-yl)morpholin-4-ylmethanone,

[4-amino-2-(3methoxyproylaminothiazol 5-yl]pridnylmethanone,

(4-amino-2-propynylaminothiazol-5-yl)pyridin-3-ylmethanone,

[4-amino-5-(pyridine-3-carbonyl)thiazol-2-ylamino]butyric acid ethyl ester,

[4-amino-2-(3,4-dichlorophenylamino)thiazol-5-yl]-(3-benzyloxyphenyl)methanone,

[4-amino-2-(4-chlorophenylamino)thiazol-5-yl]-(3-benzyloxyphenyI)methanone,

(4-amino-2-phenylaminothiazol-5-yl)-( 1-methyl-1,2,5,6-tetrahydropyridin-3-yl)methanone,

4-amino-2-phenylaminothiazole-5-carbaldehyde,

as well as any optical or geometric isomer or tautomeric form thereof including mixtures of these or a pharmaceutically acceptable salt thereof.

Some of the compounds are novel per se and constitute a further aspect of the invention.

Thus, the present invention also relates to a compound of general formula (la):

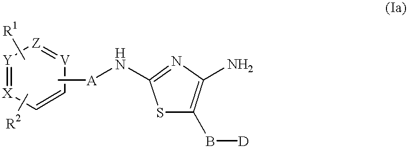

wherein

A is C₁₋₆-alkylene or —C(═O)—,

R¹, R², X, Y. Z, V, B and D are as defined for formula (I) or (I′),

with the provisos that when

X, Y, Z and V are all ═CH—, A is ethylene, B is —C(═O)—, R¹ is hydrogen, D is 2-nitrophenyl, R² must not be hydrogen or 4-chloro,

X, Y, Z and V are all ═CH—, A is ethylene, B is —C(═O)—, R¹ is hydrogen, D is 2-methoxy-phenyl, R² must not be 4-chloro,

X, Y, Z and V are all ═CH—, A is methylene, B is —C(═O)—, R¹ is hydrogen, R² is 4-methoxy, D must not be 5-chlorobenzofuran-2-yl or 2,5-dimethylthiophen-3-yl,

X, Y, Z and V are all ═CH—, A is methylene, B is —C(═O)—, R¹ and R² are both hydrogen, D must not be methyl, as well as any optical or geometric isomer or tautomeric form thereof including mixtures of these or a pharmaceutically acceptable salt thereof.

In the formula (Ia), A is preferably C₁₋₆-alkylene.

In the formula (Ia), preferred embodiments of R¹, R², X, Y, Z, V, B and D, respectively, are the same as defined above for formula (I).

Furthermore, the present invention relates to a compound of the general formula (Ib):

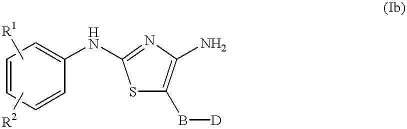

wherein

D is

hydroxy, halogen, cyano, nitro, —NR¹²R¹³, —N(R¹²)OR¹³, —C(═O)NR¹²R¹³, —OC(═O)NR¹²R¹³, —OCH₂C(═O)NR¹²R¹³, C₁₋₆-alkoxy, —C(═O)OR¹², —C(═O)R¹², —NHC(═O)R¹² —CHF₂, —CF₃, —OCF₃, —OCHF₂, —OCH₂CF₃, —OCF₂CHF₂, —SCF₃, —SR¹², —S(═O)R¹², —S(═O)₂R¹², —S(═O)₂NH₂,

wherein R¹² and R¹³ which may be the same or different independently are selected from hydrogen and C₁₋₆-alkyl or R¹² and R¹³ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR¹⁴R¹⁵, —C(═O)NR¹⁴R¹⁵, —OC(═O)NR¹⁴R¹⁵, —OCH₂C(═O)NR¹⁴R¹⁵ C₁₋₆-alkoxy, —C(═O)OR¹⁴, —C(═O)R¹⁴, —NHC(═O)R¹⁴, —CHF₂, —CF₃, —OCF₃, —OCHF₂, —OCH₂CF₃, —OCF₂CHF₂, —SCF₃, —SR¹⁴, —S(═O)R¹⁴, —S(═O)₂R¹⁴, —S(═O)₂NH₂,

wherein R¹⁴ and R¹⁵ which may be the same or different independently are selected from hydrogen and C₁₋₆-alkyl or R¹⁴ and R¹⁵ together with the nitrogen atom to which they are attached form a 3 to 8 membered cyclic ring optionally containing one or two further heteroatoms selected from oxygen, sulfur and nitrogen,

C₃₋₈-cycloalkyl, aryl-C₁₋₆-alkoxy, naphthyl, benzothiophenyl,

which may optionally be substituted with one to three substituents selected from

hydroxy, halogen, cyano, nitro, —NR¹⁶R¹⁷, —C(═O)NR¹⁶R¹⁷, —OC(═O)NR¹⁶R¹⁷ —OCH₂C(═O)NR¹⁶R¹⁷, C₁₋₆-alkoxy, —C(═O)OR¹⁶, —C(═O)R¹⁶ NHC(O)R¹⁶, —CHF₂, —CF₃, —OCF₃, —OCHF₂, —OCH₂CF₃, —OCF₂CHF₂, —SCF₃, —SR¹⁶, —S(═O)R¹⁶, —S(═O)₂R¹⁶, —S(═O)₂NH₂,

wherein R¹⁶ and R¹⁷ which may be the same or different independently are selected from hydrogen and C₁₋₆-alkyl or R¹⁶ and R¹⁷ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR¹⁸R¹⁹, —C(═O)NR¹⁸R¹⁹, —OC(═O)NR¹⁸R¹⁹, —OCH₂C(═O)NR¹⁸R¹⁹, C₁₋₆-alkoxy, —C(═O)OR¹⁸, —C(═O)R¹⁸, —NHC(═O)R¹⁸, —CHF₂, —CF₃, —OCF₃, —OCHF₂, —OCH₂CF₃, —OCF₂CHF₂, —SCF₃, —SR¹⁸, —S(═O)R¹⁸, —S(═O)₂R¹⁸, —S(═O)₂NH₂,

wherein R¹⁸ and R¹⁹ which may be the same or different independently are selected from hydrogen and C₁₋₆-alkyl or R¹⁸ and R¹⁹ together with the nitrogen atom to which they are attached form a 3 to 8 membered cyclic ring optionally containing one or two further heteroatoms selected from oxygen, sulfur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkylcarbonyl, heteroaroyl, C₃₋₈-heterocyclylcarbonyl, —O-aryl, —O—C₃₋₈-cycloalkyl, —O-heteroaryl, —O—C₃₋₈-heterocyclyl, —S-aryl, —S—C₃₋₈-cycloalkyl, —S-hetero-aryl, —S—C₃₋₈-heterocyclyl,

which may optionally be substituted with one to three substituents selected from hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆-alkyl, C₂₋₆-alkenyl, C₂₋₆-alkynyl, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,

wherein R²⁰ and R²¹ which may be the same or different independently are selected from hydrogen and C₁₋₆-alkyl or R²⁰ and R²¹ together with the nitrogen atom to which they are attached form a 3 to 8 membered cyclic ring optionally containing one or two further heteroatoms selected from oxygen, sulfur and nitrogen,

R¹, R² and B are as defined for formula (I),

with the provisos that when

R¹ and R² are both hydrogen, B is —C(═O)—, D must not be methyl, ethyl, cyclopropyl, methoxy, ethoxy or amino,

R¹ is hydrogen, R² is 4-dimethylamino, B is —C(═O)—, D must not be ethoxy, tert-butoxy, benzyloxy, 3-methylbenzothiophen-2-yl or 3-methyl-6-chlorobenzothiophen-2-yl,

R¹ is hydrogen, R² is 4-aminosulfonyl, B is —C(═O)—, D must not be 3-methylbenzothiophen-2-yl,

R¹ is hydrogen, R² is 4-chloro, B is —C(═O)—, D must not be methyl,

R¹ is hydrogen, R² is 3-methoxycarbonyl, B is —C(═O)—, D must not be naphthyl,

R¹ and R² are both hydrogen, B is a valence bond, D must not be cyano,

as well as any optical or geometric isomer or tautomeric form thereof including mixtures of these or a pharmaceutically acceptable salt thereof.

In a preferred embodiment of the compounds of the formula (Ib)

D is

hydroxy, halogen, cyano, nitro, —NR¹²R¹³, —C(═O)NR¹²R¹³, —OC(═O)NR¹²R¹³, —OCH₂C(═O)NR¹²R¹³, C₁₋₆-alkoxy, —C(═O)OR¹², —C(═O)R¹², —NHC(═O)R¹², —CHF₂, —CF₃, —OCF₃, —OCHF₂, —OCH₂CF₃, —OCF₂CHF₂, —SCF₃, —SR¹², —S(═O)R¹², —S(═O)₂R¹², —S(═O)₂NH₂,

wherein R¹² and R¹³ which may be the same or different independently are selected from hydrogen and C₁₋₆-alkyl or R¹² and R¹³ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR¹⁴R¹⁵, —C(═O)NR¹⁴R¹⁵, —OC(═O)NR¹⁴R¹⁵, —OCH₂C(═O)NR¹⁴R¹⁵, C₁₋₆-alkoxy, —C(═O)OR¹⁴, —C(═O)R¹⁴, —NHC(═O)R¹⁴, —CHF₂, —CF₃, —OCF₃, —OCHF₂, —OCH₂CF₃, —OCF₂CHF₂, —SCF₃, —SR¹⁴, —S(═O)R¹⁴, —S(═O)₂R¹⁴, —S(═O)₂NH₂,

wherein R¹⁴ and R¹⁵ which may be the same or different independently are selected from hydrogen and C₁₋₆-alkyl or R¹⁴ and R¹⁵ together with the nitrogen atom to which they are attached form a 3 to 8 membered cyclic ring optionally containing one or two further heteroatoms selected from oxygen, sulfur and nitrogen,

C₃₋₈-cycloalkyl, aryl-C₁₋₆-alkoxy, naphthyl, benzothiophenyl,

which may optionally be substituted with one to three substituents selected from

hydroxy, halogen, cyano, nitro, —NR¹⁶R¹⁷, —C(═O)NR¹⁶R¹⁷, —OC(═O)NR¹⁶R¹⁷, —CH₂C(═O)NR¹⁶R¹⁷, C₁₋₆-alkoxy, —C(═O)OR¹⁶, —C(═O)R¹⁶, —NHC(═O)R¹⁶, —CHF₂, —CF₃, —OCF₃, —OCHF₂, —OCH₂CF₃, —OCF₂CHF₂, —SCF₃, —SR¹⁶, —S(═O)R¹⁶, —S(═O)₂R¹⁶, —S(═O)₂NH₂,

wherein R¹⁶ and R¹⁷ which may be the same or different independently are selected from hydrogen and C₁₋₆-alkyl or R¹⁶ and R¹⁷ together with the nitrogen atom to which they are attached form a 3 to 8 membered cyclic ring optionally containing one or two further heteroatoms selected from oxygen, sulfur and nitrogen,

C₁₋₆-alkyl, C₂₋₆-alkenyl, C₂₋₆-alkynyl,

which may optionally be substituted with one or two substituents selected from hydroxy, halogen, cyano, nitro, —NR¹⁸R¹⁹, —C(═O)NR¹⁸R¹⁹, —OC(═O)NR¹⁸R¹⁹, —OCH₂C(═O)NR¹⁸R¹⁹, C₁₋₆-alkoxy, —C(═O)OR¹⁸, —C(═O)R¹⁸, —NHC(═O)R¹⁸, —CHF₂, —CF₃, —OCF₃, —OCHF₂, —OCH₂CF₃, —OCF₂CHF₂, —SCF₃, —SR¹⁸, —S(═O)R¹⁸, —S(═O)₂R¹⁸, —S(═O)₂NH₂,

wherein R¹⁸ and R¹⁹ which may be the same or different independently are selected from hydrogen and C₁₋₆-alkyl or R¹⁸ and R¹⁹ together with the nitrogen atom to which they are attached form a 3 to 8 membered cyclic ring optionally containing one or two further heteroatoms selected from oxygen, sulfur and nitrogen,

aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkylcarbonyl, heteroaroyl, C₃₋₈-heterocyclylcarbonyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,

which may optionally be substituted with one to three substituents selected from hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆-alkyl, C₂₋₆-alkenyl, C₂₋₆-alkynyl, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,

wherein R²⁰ and R²¹ which may be the same or different independently are selected from hydrogen and C₁₋₆-alkyl or R²⁰ and R²¹ together with the nitrogen atom to which they are attached form a 3 to 8 membered cyclic ring optionally containing one or two further heteroatoms selected from oxygen, sulfur and nitrogen.

In the formula (Ib), preferred embodiments of R¹, R² and B, respectively, are the same as defined above for formula (I).

The present invention also relates to a compound of the general formula (Ic):

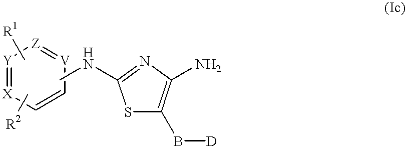

wherein one or two of X, Y, V and Z are ═N—, the rest being ═CH—,

R¹, R², B and D are as defined for formula (I) or (I′),

with the provisos that when

R¹ and R² are hydrogen, B is —C(═O)—, X, Y and V are ═CH—, and Z is ═N— in the 3-position, D must not be phenyl, 2-nitrophenyl, 2,4-dimethylphenyl, 2,4-dichlorophenyl, 2-methoxyphenyl or naphthyl,

as well as any optical or geometric isomer or tautomeric form thereof including mixtures of these or a pharmaceutically acceptable salt thereof.

In the formula (Ic), preferred embodiments of R¹, R², B and D, respectively, are the same as defined above for formula (I).

Furthermore, the present invention relates to a compound of the general formula (Id):

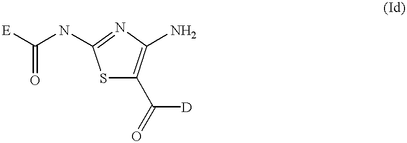

wherein E is C₁₋₆-alkyl, C₂₋₆-alkenyl, C₂₋₆-alkynyl, C₁₋₆-alkylthio, C₁₋₆-alkoxy, C₁₋₆-alkanoyloxy, —C(═O)OH or —C(═O)O—C₁₋₆-alkyl, and D is as defined for formula (I).

In a preferred embodiment of the compounds of the formula (Id), E is C₁₋₆-alkyl and D is as defined for formula (I′).

In another preferred embodiment of the compounds of the formula (Id), E is C₁₋₆-alkyl, C₁₋₆-alkoxy or —C(═O)O—C₁₋₆-alkyl.

In the formula (Id), preferred embodiments of D are the same as defined above for formula (I).

In a preferred embodiment of the invention the compound is selected from

(4-amino-2-phenylaminothiazol-5-yl)pyridin-3-yl-methanone,

(4-amino-2-phenylaminothiazol-5-yl)-(4-trifluoromethylphenyl)methanone,

(4-amino-2-phenylaminothiazol-5-yl)-(4-diethylaminophenyl)methanone,

(4-amino-2-phenylaminothiazol-5-yl)-(4-trifluoromethoxyphenyl)methanone,

4-amino-2-phenylaminothiazole-5-carboxylic acid tert-butyl ester,

1 -(amino-2-phenylaminothiazol-5-yl)-2,2-dimethylpropan-1-one,

1 -(4-amino-2-phenylaminothiazol-5-yl)propan-1-one,

(4-amino-2-phenylaminothiazol-5-yl)-(3,4-difluorophenyl)methanone,

(4-amino-2-phenylaminothiazol-5-yl)-(3-fluorophenyl)methanone,

(4-amino-2-phenylaminothiazol-5-yl)naphthalen-2-ylmethanone,

(4-amino-2-phenylaminothiazol-5-yl)biphenyl-4-ylmethanone,

(4-amino-2-phenylaminothiazol-5-yl)-(3-benzyloxyphenyl)methanone,

[4-amino-2-(4-bromophenylamino)-thiazol-5-yl]cyclopropylmethanone,

N-(4-amino-5-cyclopropanecarbonylthiazol-2-yl)benzamide,

(4-amino-2-phenylaminothiazol-5-yl)-(3,4-dichlorophenyl)methanone,

(4-amino-2-phenylaminothiazol-5-yl)-(3-methylbenzo[b]thiophen-2-yl)methanone,

(4-amino-2-phenylaminothiazol-5-yl)-(5-chlorothiophen-2-yl)methanone,

(4-amino-2-phenylaminothiazol-5-yl)-(2-methoxyphenyl)methanone,

(4-amino-2-phenylaminothiazol-5-yl)-(3-methoxyphenyl)methanone,

(4-amino-2-phenylaminothiazol-5-yl)-(4-methoxyphenyl)methanone,

(4-amino-2-phenylaminothiazol-5-yl)-(2-nitrophenyl)methanone,

(4-amino-2-phenylaminothiazol-5-yl)-(4-chloro-3-methylphenyl)methanone,

(4-amino-2-phenylaminothiazol-5-yl)-(3,4-dihydroxyphenyl)methanone,

(4-amino-2-phenylaminothiazol-5-yl)-(2,4-dimethoxyphenyl)methanone,

[4-amino-2-(4-bromo-phenylamino)thiazol-5-yl]pyridin-3-yl-methanone,

(4-amino-2-propylaminothiazol-5-yl)pyridin-3-yl-methanone,

(4-amino-2-phenylaminothiazol-5-yl)pyridin-2-y.-methanone,

(4-amino-2-phenylaminothiazol-5-yl)-pyridin-4-yl-methanone,

(4-amino-2-phenylaminothiazol-5-yl)thiophen-2-yl-methanone,

(4-amino-2-phenylaminothiazol-5-yl)-(2,6-dimethoxyphenyl)methanone,

4-amino-2-phenylaminothiazole-5-carboxylic acid methyl ester,

(4-amino-2-phenylaminothiazol-5-yl)thiophen-3-ylmethanone,

(4-amino-2-phenylaminothiazol-5-yl)-benzo[b]thiophen-3-yl-methanone,

(4-amino-2-phenylaminothiazol-5-yl)-(2,6-difluorophenyl)methanone,

(4-amino-2-phenylaminothiazol-5-yl)-(2,6-dichlorophenyl)methanone,

1-(4-amino-2-phenylaminothiazol-5-yl) ethanone,

4-amino-2-phenylaminothiazole-5-carboxylic acid methoxymethylamide,

4-amino-2-phenylaminothiazole-5-carboxylic acid amide,

(4-amino-2-phenylaminothiazol-5-yl)morpholin-4-ylmethanone,

(4-amino-2-prop-2-ynylaminothiazol-5-yl)pyridin-3-ylmethanone,

3-[4-amino-5-(pyridine-3-carbonyl)thiazol-2-ylamino]butyric acid ethyl ester,

[4-amino-2-(3,4-dichlorophenylamino)thiazol-5-yl]-(3-benzyloxyphenyl)methanone,

[4-amino-2-(34-dchlorophenylamino)thiazol-5-yl]-(3-benzyloxyphenyI)methanone,

(4-amino-2-phenylaminothiazol-5-yl)-(1-methyl-1,2,5,6-tetrahydropyridin-3-yl)methanone,

4-amino-2-phenylaminothiazole-5-carbaldehyde,

as well as any optical or geometric isomer or tautomeric form thereof including mixtures of these or a pharmaceutically acceptable salt thereof.

The compounds of the present invention may have one or more asymmetric centres and it is intended that any optical isomers, as separated, pure or partially purified optical isomers or racemic mixtures thereof are included within the scope of the invention.

Furthermore, when a double bond or a fully or partially saturated ring system is present in the molecule geometric isomers may be formed. It is intended that any geometric isomers, as separated, pure or partially purified geometric isomers or mixtures thereof are included within the scope of the invention. Likewise, molecules having a bond with restricted rotation may form geometric isomers. These are also intended to be included within the scope of the present invention.

Furthermore, some of the compounds of the present invention may exist in different tautomeric forms and it is intended that any tautomeric forms, which the compounds are able to form, are included within the scope of the present invention.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

Also intended as pharmaceutically acceptable acid addition salts are the hydrates, which the present compounds are able to form.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of the present invention may form solvates with standard low molecular weight solvents using methods well known to the person skilled in the art. Such solvates are also contemplated as being within the scope of the present invention.

The invention also encompasses prodrugs of the present compounds, which on administration undergo chemical conversion by metabolic processes before becoming active pharmacological substances. In general, such prodrugs will be functional derivatives of the present compounds, which are readily convertible in vivo into the required compound of the formula (I). Conventional procedures for the selection and preparation of suitable prodrug derivatives are described, for example, in “Design of Prodrugs”, ed. H. Bundgaard, Elsevier, 1985.

The invention also encompasses active metabolites of the present compounds. The present compounds are useful for the treatment of hyperglycemia; IGT (impaired glucose tolerance); syndrome X; Type 1 diabetes; Type 2 diabetes; conditions with dyslipidemia including diabetic dyslipidemia; and obesity. Furthermore, they may be useful for the treatment of albuminuria; cardiovascular diseases such as cardiac hypertrophy, hypertension and arteriosclerosis including atherosclerosis; gastrointestinal disorders; acute pancreatitis; and appetite regulation or energy expenditure disorders.

In addition to the GSK-3 inhibiting activity some of the present compounds have furthermore been shown to potentiate the glucose-induced stimulation of insulin secretion. They might also be expected to potentiate the glucose-induced inhibition of glucagon secretion.

Accordingly, such compounds possess a dual mechanism of action improving both the glucose disposal and insulin secretion. This dual mechanism of action makes them very attractive as antidiabetic agents, especially for the treatment of Type 2 diabetes where multipledrug therapies with different approaches to reduce hyperglycemia are often necessary.

The present compounds may also find use in the treatment and/or prevention of bipolar disorder (manic depressive syndrome), mania, Alzheimer's disease, bipolar disorder, Huntington's chorea, Parkinson's disease, amyotrophic lateral sclerosis, multiple sclerosis, leukopenia, anxiety, movement disorder, aggression, psychosis, seizures, panic attacks, hysteria or sleep disorders. Furthermore, they may be useful as contraceptives, cf WO 97/41854, and for the treatment of cancer, hair-loss and neurotraumatic diseases, such as acute stroke, cf WO 00/21927.

The novel compounds of the general formulae (Ia), (Ib), (Ic) and (Id) may also have CDK inhibiting activity and accordingly find use in the treatment and/or prevention of disorders and diseases wherein such an inhibition is beneficial.

In a preferred embodiment of the invention the present compounds are used for the manufacture of a pharmaceutical composition for the treatment and/or prevention of diseases and disorders related to GSK-3.

In a further preferred embodiment of the invention the present compounds are used for the manufacture of a pharmaceutical composition for the treatment and/or prevention of diseases and disorders wherein growth factor induced inhibition of GSK-3 is insufficient.

In another preferred embodiment of the invention the present compounds are used for the preparation of a pharmaceutical composition for the treatment and/or prevention of diseases and disorders wherein glycogen metabolism exhibits abnormalities.

In another preferred embodiment of the invention the present compounds are used for the preparation of a pharmaceutical composition for the treatment and/or prevention of diseases and disorders wherein glycogen synthase is insufficiently activated.

In a further preferred embodiment of the invention the present compounds are used for the preparation of a pharmaceutical composition for the treatment and/or prevention of diseases and disorders involving elevated blood glucose, both elevated fasting and postprandial blood glucose.

In still a further preferred embodiment of the invention the present compounds are used for the preparation of a pharmaceutical composition for the treatment and/or prevention of hyperglycemia. The present compounds are effective in lowering the blood glucose both in the fasting and postprandial stage.

In yet a further preferred embodiment of the invention the present compounds are used for the preparation of a pharmaceutical composition for the treatment and/or prevention of IGT.

In still another preferred embodiment of the invention the present compounds are used for the preparation of a pharmaceutical composition for the treatment of Type 2 diabetes. Such treatment includes ia the delaying of the progression from IGT to Type 2 diabetes as well as the delaying of the progression from non-insulin requiring Type 2 diabetes to insulin requiring Type 2 diabetes.

In a further preferred aspect of the invention the present compounds are used for the preparation of a pharmaceutical composition for the treatment and/or prevention of Type 1 diabetes. Such treatment and/or prevention are normally accompanied by insulin therapy.

Furthermore, the present compounds may be used for the preparation of a pharmaceutical composition for the treatment and/or prevention of obesity.

In a further aspect of the invention the present compounds are combined with diet and/or exercise.

In another aspect of the invention the present compounds may be used for the preparation of a pharmaceutical composition for the treatment and/or prevention of Alzheimer's disease.

In another aspect of the invention the present compounds may be used for the preparation of a pharmaceutical composition for the treatment and/or prevention of bipolar disorder.

In yet another aspect of the invention the present compounds are administered in combination with one or more further active substances in any suitable ratios. Such further active agents may be selected from antidiabetic agents, antihyperlipidemic agents, antiobesity agents, antihypertensive agents and agents for the treatment of complications resulting from or associated with diabetes. Furthermore, they may be administered in combination with one or more further pharmacologically active substances selected from agents for the treatment of Alzheimer's disease and agents for the treatment of bipolar disorder. Such combined administration may be in separate preparations or in a single preparation, as appropriate.

Suitable antidiabetics comprise insulin, GLP-1 derivatives such as those disclosed in WO 98/08871 (Novo Nordisk A/S), which is incorporated herein by reference as well as orally active hypoglycemic agents.

The orally active hypoglycemic agents preferably comprise sulfonylureas, biguanides, meglitinides, oxadiazolidinediones, thiazolidinediones, a-glucosidase inhibitors, glucagon antagonists such as those disclosed in WO 99/01423 and WO 00/39088 (Novo Nordisk A/S and Agouron Pharmaceuticals, Inc.), GLP-1 agonists such as those disclosed in WO 00/42026 (Novo Nordisk A/S and Agouron Pharmaceuticals, Inc.), potassium channel openers such as those disclosed in WO 97/26265, WO 99/03861 and WO 00/37474 (Novo Nordisk A/S), insulin sensitizers, DPP-IV inhibitors, PTPase inhibitors, inhibitors of hepatic enzymes involved in stimulation of gluconeogenesis and/or glycogenolysis, glucose uptake modulators, compounds modifying the lipid metabolism such as antihyperlipidemic agents and antilipidemic agents, compounds lowering food intake, PPAR and RXR agonists and agents acting on the ATP-dependent potassium channel of the β-cells.

In one embodiment of the invention the present compounds are administered in combination with insulin.

In a further embodiment the present compounds are administered in combination with a sulfonylureaegtolbutamide, glibenclamide, glipizide, glimepiride, glicazide or glyburide.

In another embodiment the present compounds are administered in combination with a biguanide eg metformin.

In yet another embodiment the present compounds are administered in combination with a meglitinide eg repaglinide or senaglinide.

In still another embodiment the present compounds are administered in combination with a thiazolidinedione eg troglitazone, ciglitazone, pioglitazone, rosiglitazone or the compounds disclosed in WO 97/41097, WO 97/41119, WO 97/41120, WO 00/41121 and WO 98/45202 (Dr. Reddy's Research Foundation).

Furthermore, the present compounds may be administered in combination with an insulin sensitizer eg such as those disclosed in WO 99/19313, WO 00/50414, WO 00/63191, WO 00/63192, WO 00/63193 (Dr. Reddy's Research Foundation) and WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S).

In a further embodiment the present compounds are administered in combination with an α-glucosidase inhibitor eg miglitol or acarbose.

In another embodiment the present compounds are administered in combination with an agent acting on the ATP-dependent potassium channel of the β-cells eg tolbutamide, glibenclamide, glipizide, glicazide or repaglinide.

Furthermore, the present compounds may be administered in combination with nateglinide.

In still another embodiment the present compounds are administered in combination with an antihyperlipidemic agent or antilipidemic agent eg cholestyramine, colestipol, clofibrate, gemfibrozil, lovastatin, pravastatin, simvastatin, probucol or dextrothyroxine.

In another aspect of the invention, the present compounds are administered in combination with more than one of the above-mentioned compounds eg in combination with metformin and a sulphonylurea such as glyburide; a sulphonylurea and acarbose; nateglinide and metformin; acarbose and meformin; a sulfonylurea, metformin and troglitazone; insulin and a sulfonylurea; insulin and metformin; insulin, metformin and a sulfonylurea; insulin and troglitazone; insulin and lovastatin; etc.

Furthermore, the compounds according to the invention may be administered in combination with one or more antiobesity agents or appetite regulating agents.

Such agents may be selected from the group consisting of CART (cocaine amphetamine regulated transcript) agonists, NPY (neuropeptide Y) antagonists, MC4 agonists, orexin antagonists, H3 antagonists, TNF (tumor necrosis factor) agonists, growth factors such as prolactin or placental lactogen, CRF (corticotropin releasing factor) agonists, CRF BP (corticotropin releasing factor binding protein) antagonists, urocortin agonists, β3 agonists, MSH (melanocytestimulating hormone) agonists, MCH (melanocyte-concentrating hormone) antagonists, CCK agonists, serotonin re-uptake inhibitors, mixed serotonin and noradrenergic compounds, 5HT agonists, bombesin agonists, galanin antagonists, growth hormone, growth hormone releasing compounds, TRH agonists, UCP (uncoupling protein) 2 or 3 modulators, leptin agonists, DA agonists (bromocriptin, doprexin), lipaselamylase inhibitors, PPAR modulators, RXR modulators, TR β agonists, AGRP (Agouti related protein) inhibitors and H3 histamine antagonists such as those disclosed in WO 00/42023, WO 00/63208 and WO 00164884, which are incorporated herein by reference.

In one embodiment of the invention the antiobesity agent is leptin.

In still another embodiment of the invention the antiobesity agent is dexamphetamine, amphetamine, phentermine, mazindol, phendimetrazine, diethylpropion, fenfluramine or dexfenfluramine.

In still another embodiment the antiobesity agent is sibutramine.

In a further embodiment the antiobesity agent is orlistat.

Furthermore, the present compounds may be administered in combination with one or more antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and a-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

Furthermore, the present compounds may be administered in combination with one or more agents for the treatment of Alzheimer's disease. Examples of such agents are tacrine, donepezil, haloperidol, olanzapine, quetiapine, risperidone, alprazolam, buspirone, diazepam, lorazepam, amitriptyline, bupropion, desipramine, fluoxetine, fluvoxamine, nefazodone, nortriptyline, paroxetine, sertraline and trazodone.

The present compounds may also be administered in combination with one or more agents for the treatment of bipolar disorder. Examples of such agents are lithium, valproate, divalproex, carbamazepine, antipsychotic drugs such as haloperidol and perphenazine, antianxiety agents such as lorazepam and clonazepam, antidepressants such as bupropion, fluoxetine, fluvoxamine, paroxetine, sertraline, mirtazepine, pheneizine, tranylcypromine, nefazodone, amitriptyline, desipramine, imipramine, nortriptyline and venlafaxine.

It should be understood that any suitable combination of the compounds according to the invention with one or more of the above-mentioned compounds and optionally one or more further pharmacologically active substances are considered to be within the scope of the present invention.

### PHARMACEUTICAL COMPOSITIONS

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain of from 0.05 to about 1000 mg, preferably from about 0.1 to about 500 mg, and more preferred from about 0.5 mg to about 200 mg. For parenteral routes, such as intravenous, intrathecal, intramuscular and similar administration, typically doses are in the order of about half the dose employed for oral administration.

The compounds of this invention are generally utilized as the free substance or as a pharmaceutically acceptable salt thereof. One example is an acid addition salt of a compound having the utility of a free base. When a compound of the formula (I) contains a free base such salts are prepared in a conventional manner by treating a solution or suspension of a free base of the formula (I) with a chemical equivalent of a pharmaceutically acceptable acid, for example, inorganic and organic acids. Representative examples are mentioned above. Physiologically acceptable salts of a compound with a hydroxy group include the anion of said compound in combination with a suitable cation such as sodium or ammonium ion.

For parenteral administration, solutions of the novel compounds of the formula (I) in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phosphol ipids, fatty acids, fatty acid amines, polyoxyethylene or water. Similarly, the carrier or diluent may include any sustained release material known in the art, such as glyceryl monostearate or glyceryl distearate, alone or mixed with a wax. The pharmaceutical compositions formed by combining the novel compounds of the formula (I) and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

| | | |
|---|---|---|
| Core: | | |
| Active compound (as free compound or salt thereof) | 5.0 | mg |
| Lactosum Ph. Eur. | 67.8 | mg |
| Cellulose, microcryst. (Avicel) | 31.4 | mg |
| Amberlite ® IRP88* | 1.0 | mg |
| Magnesii stearas Ph. Eur. | q.s. | |
| Coating: | | |
| Hydroxypropyl methylcellulose | approx. 9 | mg |
| Mywacett 9-40 T** | approx. 0.9 | mg |

*Polacrillin potassium NF, tablet disintegrant, Rohm and Haas.
**Acylated monoglyceride used as plasticizer for film coating.

If desired, the pharmaceutical composition of the invention may comprise the compound of the formula (I) in combination with further pharmacologically active substances such as those described in the foregoing.

The present invention is further illustrated by the following representative examples which are, however, not intended to limit the scope of the invention in any way.

### EXAMPLES

The compounds used as starting materials are either known compounds or compounds, which can be prepared by methods known per se. NMR spectra were recorded on Bruker 200 MHz and 300 MHz instruments. Mass Spectra were run on a Finnigan MAT TSQ70B as SP-MS. Flash chromatography was carried out on Merck silica gel 60 (Art 9385).

HPLC-MS Method:

Instruments:

Sciex API 100 single quadropole mass spectrometer,

Applied Biosystems 785A UV detector,

Sedex 55 evaporative light scattering detector.

Gradient: 5% - 90% acetonitrile (with 0.05% TFA) during 7.5 min. UV detection at 214 nm.

In the examples and assays the following terms are intended to have the following meanings:

DMF: N,N-dimethylformamide

DMSO: dimethyl sulphoxide

EtOAc: ethyl acetate

M.p.: melting point

TFA: trifluoroacetic acid

### Example 1

(4-Amino-2-phenylaminothiazol-5-yl)cyclopropylmethanone

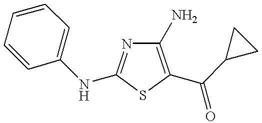

The method described by Rajasekharan K. N. et al (Synthesis, 5,1986, pp 353-355) was employed using 2-bromo-1-cyclopropylethanone and 1-phenyl-3-guanylthiourea. 2-Bromo-1-cyclopropylethanone was itself prepared using a slight modification to the literature procedure described by Calverley M. J., (Tetrahedron, 43, 20, 1987, 4609-4619). A temperature of 10-15° C. was used throughout the addition of bromine. The title compound was purified by recrystallisation from EtOAc/n-heptane to afford pink microcrystals.

M.p. 170-171° C.; ¹H NMR (300 MHz; CDCl₃): δ 0.87 (2H, m, CH₂), 1.13 (2H, m, CH₂), 1.76 (1H, m, CH), 6.55 (2H, br s, NH₂), 7.15-7.45 (5H, m, Ar—H), 8.63 (1H, s, NH).

The following compounds, unless specified otherwise, were prepared as described in example 1 using the appropriate starting materials.

### Example 2

(4-Amino-2-phenylaminothiazol-5-yl)-(4-fluorophenyl)methanone

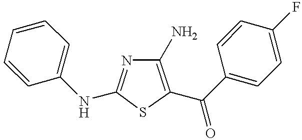

M.p. 185-186° C.

### Example 3

(4-Amino-2-phenylaminothiazol-5-yl)-(4-chlorophenyimethanone

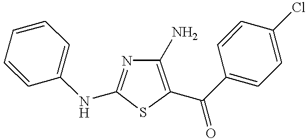

M.p. 194-197° C.

### Example 4

(4-Amino-2-phenylaminothiazol-5-yl)phenylmethanone

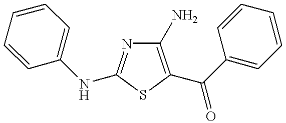

M.p. 173-177° C.

### Example 5

(4-Amino-2-phenylaminothiazol-5-yl)pyridin-3-ylmethanone

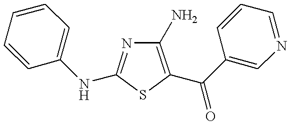

The starting material 3-(2-bromoacetyl)pyridine was first prepared as the hydrobromide salt, according to Dornow et al, (Chem. Ber., 84,1951, p 147). The title compound was prepared in the usual manner, except that an extra equivalent of triethylamine was used.

M.p. 227-228° C.; ¹H NMR (300 MHz; DMSO-d₆): δ7.10 (1H, t, Ar—H), 7.38 (2H, t, Ar—H), 7.52 (1H, dd, Ar—H), 7.62 (2H, d, Ar—H), 8.04 (1H, dd, Ar—H), 8.30 (2H, br s, NH₂), 8.68 (1H, dd, Ar—H), 8.77 (1H, d, Ar—H), 10.88 (1H, s, NH).

### Example 6

(4-Amino-2-phenylaminothiazol-5-yl)-(4-trifluoromethylphenyl)methanone

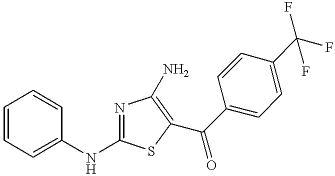

M.p. 201-205° C.; MS (EI): m/z 363 [M⁺].

### Example 7

(4-Amino-2-phenylaminothiazol-5-yl)-(4-diethylaminophenyl)methanone

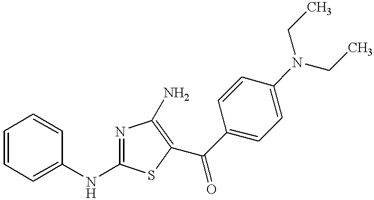

M.p. 214-216° C.; MS (EI): m/z 366 [M⁺].

### Example 8

(4-Amino-2-phenylaminothiazol-5-yl)-(4-trifluoromethoxyphenyl)methanone

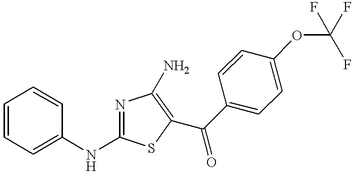

M.p. 170-173° C.

### Example 9

4-Amino-2-phenylaminothiazole-5-carboxylic acid tert-butyl ester

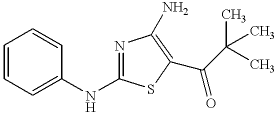

MS (El): m/z 291 [M⁺].

### Example 10

1-(4-Amino-2-phenylaminothiazol-5-yl)-2,2-dimethylpropan-1-one

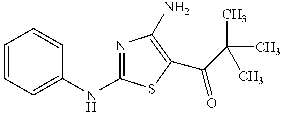

M.p. 178-181° C.; MS (EI): m/z 275 [M⁺].

### Example 11

1-(4-Amino-2-phenylaminothiazol-5-yl)propan-1-one

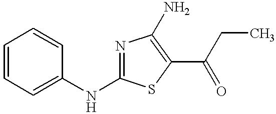

M.p. 136-139° C.; MS (EI): m/z 247 [M⁻].

### Example 12

(4-Amino-2-phenylaminothiazol-5-yl)-(3,4-difluorophenyl)methanone

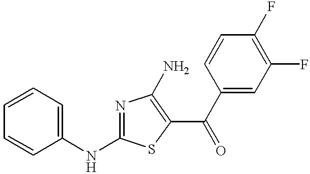

M.p. 178-180° C.; MS (EI): m/z 331 [M⁺].

### Example 13

(4-Amino-2-phenylaminothiazol-5-yl)-(3-fluorophenyl)methanone

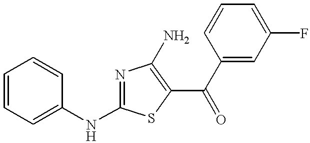

M.p. 161-164° C.; MS (EI): m/z 313 [M⁺].

### Example 14

(4-Amino-2-phenylaminothiazol-5-yl)naphthalen-2-ylmethanone

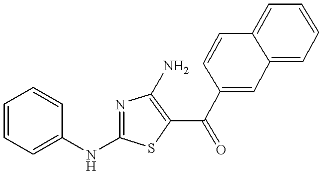

M.p. 227-229° C.; MS (EI): m/z 345 [M⁺].

### Example 15

(4-Amino-2-phenylaminothiazol-5-yl)biphenyl-4-ylmethanone

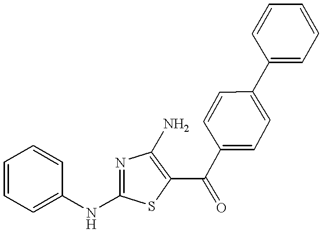

M.p. 224-225° C.; MS (EI): m/z 370 [M⁺].

### Example 16

(4-Amino-2-phenylaminothiazol-5-yl)-(3-benzyloxyphenyl)methanone

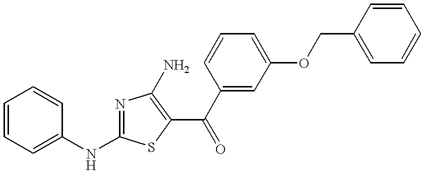

M.p. 121-124° C.; ¹H NMR (300 MHz; DMSO-d₆): δ5.17 (2H, s, CH₂), 7.03-7.49 (12H, m, Ar—H), 7.62 (2H, d, Ar—H), 8.20 (2H, br s, NH₂), 10.78 (1 H, s, NH).

### Example 17

[4-Amino-2-(4-bromophenylamino)thiazol-5-yl]cyclopropylmethanone

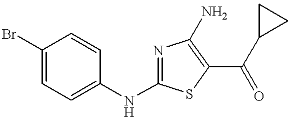

M.p. 204-206° C.; MS (EI): m/z 339 [M⁺].

### Example 18

(4-Amino-2-phenylaminothiazol-5-yl)-(3,4-dichlorophenyl)methanone

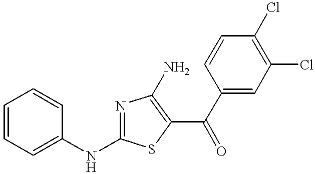

M.p. 195-198° C.

Microanalysis for C₁₆H₁₁N₃Cl₂OS:

Calc: C, 52.76%; H, 3.04%; N, 11.54%;

Found: C, 52.57%; H, 3.19%; N, 11.19%.

### Example 19

(4-Amino-2-phenylaminothiazol-5-yl)-(3-methylbenzo[b]thiophen-2-yl) methanone

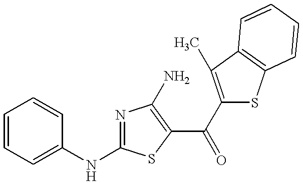

M.p. 165-167° C.; MS (EI): m/z 365 [M⁺].

### Example 20

4-Amino-2-phenylamino-5-(4-nitrophenyl)thiazole

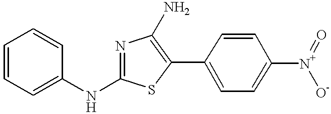

M.p. 217-219° C.; MS (EI): m/z 312 [M⁺]; ¹H NMR (300 MHz; DMSO-d₆): δ6.45 (2H, br s, NH₂), 7.03 (1H, t, Ar—H), 7.35 (2H, t, Ar—H), 7.43 (2H, d, Ar—H), 7.66 (2H, d, Ar—H), 8.07 (2H, d, Ar—H), 10.50 (1H, s, NH).

### Example 21

(4-Amino-2-phenylaminothiazol-5-yl)-(5-chlorothiophen-2-yl)methanone

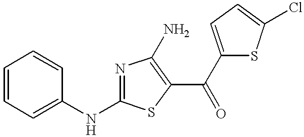

M.p. 167-169° C.; MS (EI): m/z 335 [M⁺]; ¹H NMR (300 MHz; DMSO-d₆): δ7.11 (1H, t, Ar—H), 7.23 (1H, d, Ar—H), 7.39 (2H, t, Ar—H), 7.42 (1 H, d, Ar—H), 7.66 (2H, d, Ar—H), 8.36 (2H, br s, NH₂), 10.98 (1H, s, NH).

### Example 22

(4-Amino-2-phenylaminothiazol-5-yl)-(2-methoxyphenvl)methanone

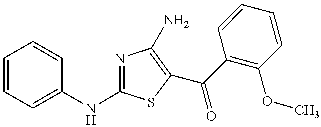

M.p. 196-200° C.; MS (EI): m/z 325 [M⁺].

### Example 23

(4-Amino-2-phenylaminothiazol-5-yl)-(3-methoxyphenyl)methanone

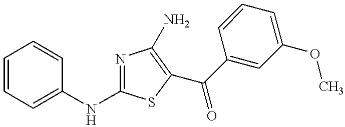

M.p. 174-175° C.; MS (EI): m/z 325 [M⁺].

### Example 24

(4-Amino-2-phenylaminothiazol-5-yl)-(4-methoxyphenyl)methanone

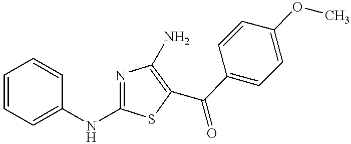

M.p. 221-223° C.; MS (EI): m/z 325 [M⁺].

### Example 25

(4-Amino-2-phenylaminothiazol-5-yl)-(2-nitrophenyl)methanone

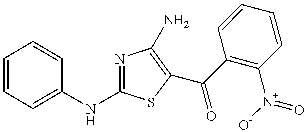

M.p. 117-119° C.; MS (EI): m/z 340 [M⁺].

### Example 26

(4-Amino-2-phenylaminothiazol-5-yl)-(3-nitrophenyl)methanone

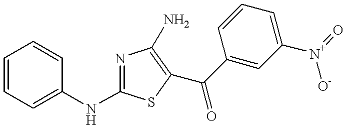

M.p. 160-163° C.; MS (EI): m/z 340 [M⁺].

### Example 27

(4-Amino-2-phenylaminothiazol-5-yl)-(4-nitrophenyl)methanone

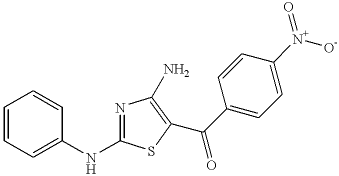

M.p. 206-208° C.; MS (EI): m/z 340 [M⁺].

### Example 28

(4-amino-2-phenylaminothiazol-5-yl)-(4-chloro-3-methylphenyl)methanone

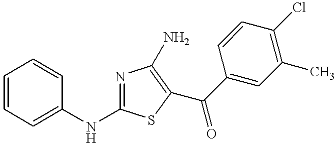

M.p. 173-175° C.; MS (EI): m/z 342 [M⁺].

### Example 29

(4-Amino-2-phenylaminothiazol-5-yl)-(3,4-dihydroxyphenyl)methanone

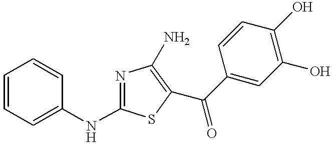

M.p. 222-224° C.

### Example 30

(4-Amino-2-phenylaminothiazol-5-yl)-(2,4-dimethoxyphenyl)methanone

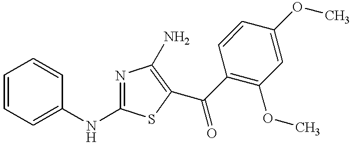

M.p. 196-198° C.; MS (EI): m/z 355 [M⁺].

### Example 31

[4-Amino-2-(4-bromo-phenylamino)thiazol-5-yl]pyridin-3-yl-methanone

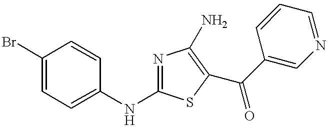

HPLC-MS (ESI): m/z 377 [M+H]⁺; Rₜ=4.16 min.

### Example 32

(4-Amino-2-propylaminothiazol-5-yl)pyridin-3-yl-methanone

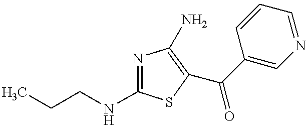

M.p. 205-206° C.; ¹H NMR (300 MHz; DMSO-d₆): δ0.88 (3H, t, CH₃), 1.57 (2H, m, CH₂), 3.23 (2H, br, CH₂), 7.48 (1H, dd, Ar—H), 7.98 (1H, dd, Ar—H), 8.64 (1H, dd, Ar—H), 8.80 (1H, d, Ar—H), 8.45 (1H, s, NH₂), 8.77 (1H, s, NH); HPLC-MS (ESI): m/z 263 [M+H]⁺; Rₜ=2.95 min.

### Example 33

(4-Amino-2-phenylaminothiazol-5-yl)pyridin-2-yl-methanone

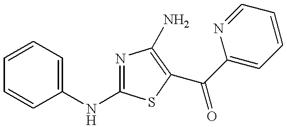

M.p. 185-186° C.; MS (EI): m/z 296 [M]⁺.

### Example 34

(4-Amino-2-phenylaminothiazol-5-yl)-pyridin-4-yl-methanone

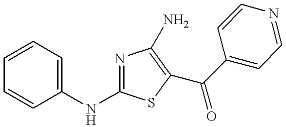

M.p. 250° C; MS (EI): m/z296 [M]⁺.

### Example 35

(4-Amino-2-phenylaminothiazol-5-yl)-(2,4,6-trimethylphenyl)methanone

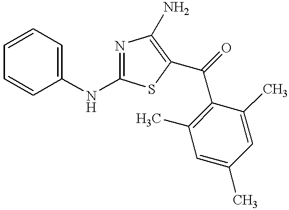

M.p. 198-200° C.; MS (EI): m/z 337 [M]⁺.

### Example 36

(4-Amino-2-phenylaminothiazol-5-yl)thiophen-2-yl-methanone

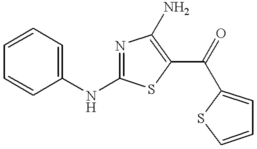

The starting material, 2-bromo-1-thiophen-2-ylethanone was first prepared, according to King, L. C. et al (J. Org. Chem., 1964, 29, pp 3459-3461). The title compound was prepared in the usual manner.

M.p. 84-85° C.

### Example 37

(4-Amino-2-phenylaminothiazol-5-yl)-(2,6-dimethoxyphenyl)methanone

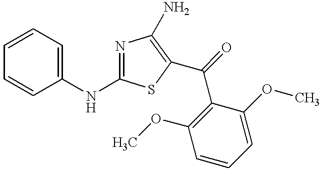

The starting material, 2-bromo-1-(2,6-dimethoxyphenyl)ethanone was first prepared, according to King, L. C. et al (J. Org. Chem., 1964, 29, pp 3459-3461). The title compound was prepared in the usual manner.

M.p. 224-226° C.

Microanalysis for C₁₈H₁₇N₃O₃S:

Calc: C, 60.83%; H, 4.82%; N, 11.82%;

Found: C, 60.82%; H, 4.89%; N, 11.70%.

### Example 38

4-Amino-2-phenylaminothiazole-5-carboxylic acid methyl ester

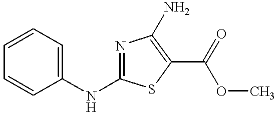

M.p. 173-174° C.; ¹H NMR (200 MHz; DMSO-d₆): δ3.64 (3H, s, CH₃), 6.94 (2H, s, NH₂), 7.05 (1H, t, Ar—H), 7.35 (2H, t, Ar—H), 7.61 (2H, d, Ar—H), 10.57 (1H, s, NH).

### Example 39

(4-Amino-2-phenylaminothiazol-5-yl)thiophen-3-ylmethanone

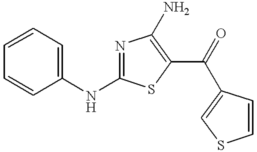

The starting material, 2-bromo-1-thiophen-3-ylethanone was first prepared, according to King, L. C. et al (J. Org. Chem., 1964, 29, pp 3459-3461). The title compound was prepared in the usual manner.

M.p. 171-172° C.; ¹H NMR (300 MHz; DMSO-d₆): δ7.08 (1H, t, Ar—H), 7.38 (2H, t, Ar—H), 7.43 (1H, dd, Ar—H), 7.60-7.68 (3H, m, Ar—H), 8.00 (1H, dd, Ar—H), 8.18 (2H, bs, NH₂), 10.83 (1H, s, NH).

### Example 40

(4-Amino-2-phenylaminothiazol-5-yl)-benzofblthiophen-3-yl-methanone

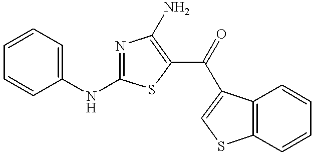

The starting material, 2-bromo-1-(2,3-dihydrobenzo[b]thiophen-3-yl)ethanone was first prepared, according to King, L. C. et al (J. Org. Chem., 1964, 29, pp 3459-3461). The title compound was prepared in the usual manner.

M.p. 211-212° C.

Microanalysis for C₁₈H₁₃N₃OS₂:

CaIc: C, 61.52%; H, 3.73%; N, 11.96%;

Found: C, 61.29%; H, 3.81%; N, 11.97%.

### Example 41

(4-Amino-2-phenylaminothiazol-5-yl)-(2,6-difluorophenyl)methanone

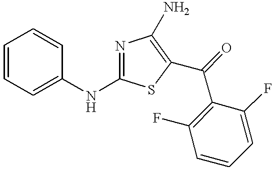

The starting material, 2-bromo-1-(2,6-difluoro-phenyl)ethanone was first prepared, according to King, L. C. et al (J. Org. Chem., 1964, 29, pp 3459-3461). The title compound was prepared in the usual manner.

M.p. 183-184 ° C.; HPLC-MS (ESI): m/z 332 [M+H]⁺; Rₜ=5.20 min.

### Example 42

(4-Amino-2-phenylaminothiazol-5-yl)-(2,6-dichlorophenyl)methanone

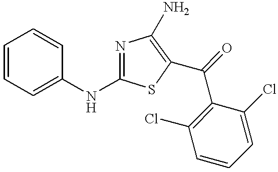

The starting material, 2-bromo-1-(2,6-dichloro-phenyl)ethanone was first prepared, according to King, L. C. et al (J. Org. Chem., 1964, 29, pp 3459-3461). The title compound was prepared in the usual manner.

M.p. 245-246 ° C.; HPLC-MS (ESI): m/z 365 [M+H]⁺; Rₜ=5.38 min.

### Example 43

4-Amino-2-phenylaminothiazole-5-carbonitrile

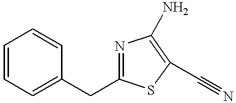

¹H NMR (300 MHz; DMSO-d₆): δ6.93 (2H, s, NH₂), 7.04 (1H, t, Ar—H), 7.34 (2H, t, Ar—H), 7.58 (2H, m, Ar—H), 10.65 (1H, s, NH); HPLC-MS (ESI): m/z 217 [M+H]³⁰ ; Rₜ=4.48 min.

### Example 44

1-(4-Amino-2-phenylaminothiazol-5-yl)ethanone

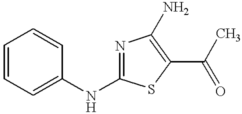

¹H NMR (300 MHz; DMSO-d₆): δ3.32 (3H, s, CH₃), 7.06 (1H, t, Ar—H), 7.36 (2H, t, Ar—H), 7.61 (2H, m, Ar—H), 7.70 (2H, s, NH₂), 10.68 (1H, s, NH).

### Example 45

4-Amino-2-phenylaminothiazole-5-carboxylic acid phenylamide

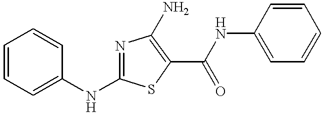

M.p. 75° C.; ¹H NMR (300 MHz; DMSO-d₆) δ6.99 (1H, t, Ar—H), 7.05 (1H, t, Ar—H), 7.10 (2H, s, NH₂), 7.26 (2H, t, Ar—H), 7.36 (2H, t, Ar—H), 7.62 (4H, m, Ar—H), 8.92 (1H, s, NH), 10.53 (1H, s, NH); HPLC-MS (ESI): m/z 311 [M+H]⁺; R=4.77 min.

### Example 46

4-Amino-2-phenyiaminothiazole-5-carboxylic acid methoxymethylamide

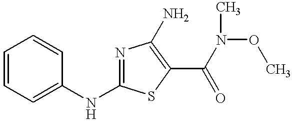

M.p. 152-153° C.; ¹H NMR (300 MHz; DMSO-d₆) δ3.11 (3H, s, CH₃), 3.68 (3H, s, CH₃) 7.02 (1H, t, Ar—H), 7.32 (4H, t, Ar—H and NH₂), 7.63 (2H, dd, Ar—H), 10.43 (1H, s, NH); HPLC-MS (ESI): m/z 279 [M+H]⁺; R=4.42 min.

### Example 47

[4-Amino-2-(pyridin-3-ylamino)thiazol-5-yl]phenylmethanone

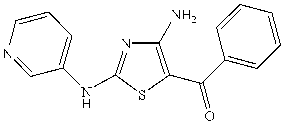

The method described by Gewald K. et al (J. Prakt. Chem., 35, 1967, pp 97-104) was employed using phenacyl bromide and pyridine-3-isothiocyanate. The title compound was obtained after chromatography using ethyl acetate as eluant. HPLC-MS (ESI): m/z 297 [M+H]⁺; Rₜ=3.63 min.

The following compounds, unless specified otherwise, were prepared as described in example 47 using the appropriate starting materials.

### Example 48

4-Amino-2-phenylaminothiazole-5-carboxvlic acid amide

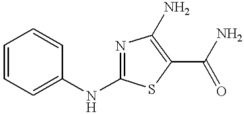

An intermediate, S-(acetamido)-N″-cyano-NΔ-phenylisothiourea was isolated when the aforementioned method of Gewald was employed:

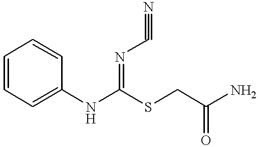

The crude intermediate was refluxed overnight in methanol with 2 equivalents of 10 triethylamine. The title compound was obtained on cooling and addition of water.

HPLC-MS (ESI): m/z 235 [M+H]⁺; Rₜ=2.80 min.

### Example 49

(4-Amino-2-phenylaminothiazol-5-yl)morpholin-4-ylmethanone

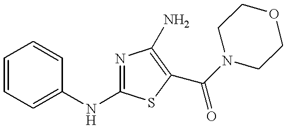

The title compound was obtained in an analogous fashion to Example 48.

M.p. 212-213° C.;

HPLC-MS (ESI): m/z 305 [M+H]⁺; Rₜ=3.43 min.

### Example 50

[4-Amino-2-(3-methoxypropylamino)thiazol-5-yl]pvridin-3-ylmethanone

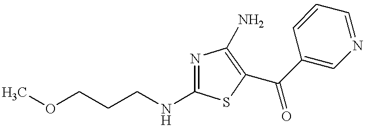

¹H NMR (300 MHz; CDCl₃): δ1.93 (2H, m, CH₂), 3.35 (3H, s, CH₃), 3.40 (2H, dd, CH₂), 3.55 (2H, dt, CH₂), 6.99 (1H, b, NH), 7.38 (1H, ddd, Ar—H), 8.05 (1H, ddd, Ar—H), 8.64 (1 H, dd, Ar—H), 8.89 (1H, d, Ar—H); HPLC-MS (ESI): m/z 293 [M+H]⁺; Rₜ=2.77 min.

### Example 51

(4-Amino-2-prop-2-ynylaminothiazol-5-yl)pyridin-3-ylmethanone

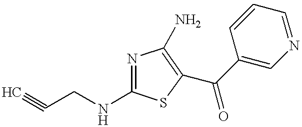

¹H NMR (300 MHz; DMSO-d₆): δ3.28 (1H, s, CH), 4.12 (2H, bs, CH₂), 7.48 (1H, dd, Ar—H), 7.98 (1H, dd, Ar—H), 8.00-8.50 (2H, bs, NH₂), 8.65 (1H, d, Ar—H), 8.89 (1H, s, Ar—H), 9.06 (1H, s, NH); HPLC-MS (ESI): m/z 259 [M+H]⁺; Rₜ=2.85 min.

### Example 52

3-[4-Amino-5-(pyridine-3-carbonyl)thiazoi-2-ylamino]butyric acid ethyl ester

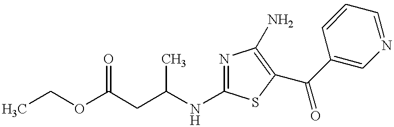

¹H NMR (300 MHz; DMSO-d₆): δ1.13-1.24 (6H, m, 2×CH₃), 2.50-2.68 (3H, m, CH and CH₂), 7.48 (1H, dd, Ar—H), 7.98 (1H, dt, Ar—H), 8.00-8.50 (2H, b, NH₂), 8.63 (1H, dd, Ar—H), 8.72 (1H, b, NH), 8.79 (1H, s, Ar—H).

### Example 53

[4-Amino-2-(3,4-dichlorophenylamino)thiazol-5-yl]-(3-benzyloxyphenyl)methanone

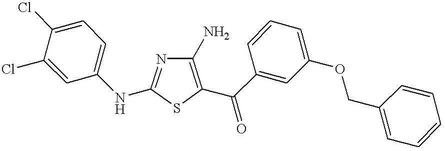

¹H NMR (300 MHz; DMSO-d₆): δ5.17 (2H, s, CH₂), 7.15-7.65 (11H, m, Ar—H), 8.12 (1H, d, Ar—H), 8.28 (2H, bs, NH₂), 10.99 (1H, s, NH₂); HPLC-MS (ESI): m/z 471 [M+H]⁺; Rₜ=8.08 min.

### Example 54

[4-Amino-2-(4-chlorophenylamino)thiazol-5-yl]-(3-benzyloxyphenyl)methanone

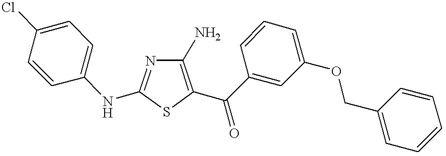

M.p. 173-174° C.; ¹H NMR (200 MHz; DMSO-d₆): δ5.16 (2H, s, CH₂), 7.15-7.65 (13H, m, Ar—H), 8.23 (2H, bs, NH₂), 10.89 (1H, s, NH).

### Example 55

(4-Amino-2-phenylaminothiazol-5-yl)-(1-methyl-1,2,5,6-tetrahydropyridin-3-yl)methanone

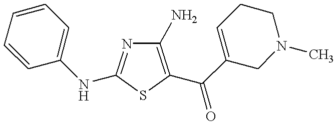

To a suspension of (4-amino-2-phenylaminothiazol-5-yl)pyridin-3-ylmethanone (example 5) (100 mg, 0.33 mmol) in acetone (20 ml) methyliodide (0.5 ml) was added. The reaction mixture was stirred at room temperature for 2 days, then evaporated In vacuo. The crude compound was resuspended in methanol (20 ml) and treated with sodium borohydride (200 mg). The reaction mixture was evaporated. Water (50 ml) was added and the water phase was extracted with ethyl acetate (4×30 ml). The organic phase was dried over magnesium sulphate and evaporated. The crude compound was purified by column chromatography on silica with ethyl acetate/methanol/conc. ammonium hydroxide (3:1:0.03) as eluent to give the free base of the title compound. The free base was crystallised as the oxalate salt from acetone.

M.p. 193-94° C.

### Example 56

4-Amino-2-phenvlaminothiazole-5-carbaldehyde

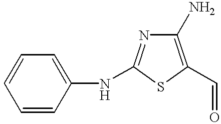

Step A:

Phenylthiocarbamide (24.32 g, 160.0 mmol) was dissolved in a mixture of toluene (230 ml) and DMF (45 ml). Ethyl bromoacetate (16.8 ml, 160 mmol) was added and the reaction mixture was stirred for 4 hours at room temperature. Triethylamine (22.3 ml, 160 mmol) was added and the reaction mixture was stirred overnight at room temperature. The toluene was evaporated and water was added. The precipitated compound was filtered, washed with water and dried to give 27.0 g (87%) of 2-phenylaminothiazol-4-one.

Step B:

2-Phenylaminothiazol-4-one (12.48 g, 65 mmol) was added to a Vielsmeier-Hack reagent, prepared from DMF (21 ml) and phosphorusoxytrichloride (21 ml). The reaction mixture was heated at 90° C. for 1 hour, and then carefully added to an ice-water mixture and stirred at room temperature for 4 hours. The precipitated compound was filtered, washed with water and dried. The crude compound was dissolved in acetonitrile and a solution of potassium carbonate (12.3 g) in water (125 ml) was added. The reaction mixture was stirred for another 2 hours and water (200 ml) was added. The precipitated compound was filtered, washed with water and dried to give 6.08 g (40%) of 4-chloro-2-phenylaminothiazole-5-carbaldehyde.

Step C:

To a solution of sodium azide (130 mg, 2.0 mmol in DMSO (10 ml), 4-chloro-2-phenylamino-thiazole-5-carbaldehyde (238 mg, 1.0 mmol) was added. The reaction mixture was stirred at room temperature overnight. Water was added to the reaction mixture and the precipitated compound was filtered and washed with water. The crude azide was suspended in methanol (30 ml) and sodium hydrosulphide hydrate (370 mg, 5 mmol) dissolved in water (5 ml) was added. The reaction mixture was stiired at room temperature for 1 hour and then evaporated to a 5 ml volume. Water was added and the precipitated compound was filtered, washed with water and dried to give the title compound in 120 mg (52%) yield. M.p. 212-13° C.

### Example 57

[4-Amino-2-(4-chlorophenylamino)thiazol-5-yl]phenylmethanone

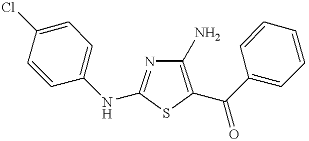

This compound was purchased from Bionet Research Ltd, Camelford, UK.

### Example 58

(4-Amino-2-ethylaminothiazol-5-yl)phenylmethanone

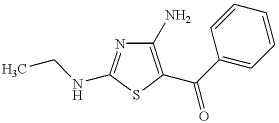

This compound was purchased from SPECS and BioSPECS B.V., Rijswijk, The Netherlands.

The following compounds are also within the scope of the present invention:

| | |
|---|---|
| (4-amino-2-phenylaminothiazol-5-yl)oxo- | 1-(4-amino-2-phenylaminothiazol-5-yl)-2,2,2- |
| acetic acid ethyl ester | trifluoroethanone |
| 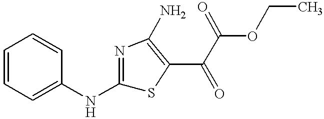 | 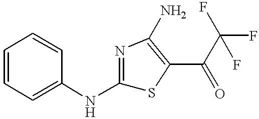 |
| 4-amino-2-phenylaminothiazole-5-carboxylic | N-(4-amino-5-cyclopropanecarbonylthiazol-2- |
| acid benzyl ester | yl)benzamide |
| 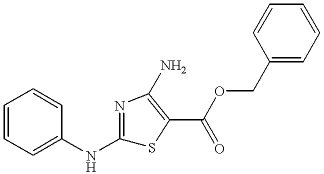 | 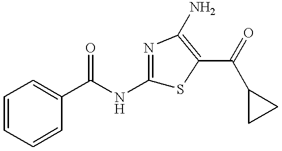 |
| (4-amino-2-phenylaminothiazol-5-yl)oxo- | 4-amino-2-phenylaminothiazole-5-carboxylic |
| acetic acid | acid |
| 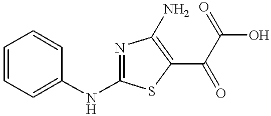 | 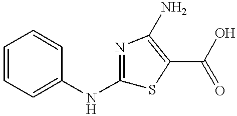 |
| (4-amino-2-phenylaminothiazol-5-yl)- | 4-amino-2-phenylamino-5-(4-bromophenyl)- |
| (phenyl)sulphone | thiazole |
| 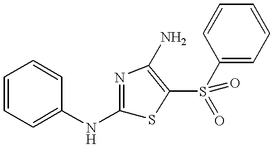 | 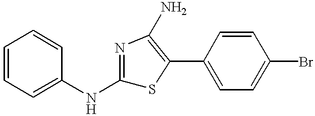 |
| 4-amino-2-phenylamino-5-(2,4-difluoro- | (4-amino-2-phenylaminothiazol-5-yl)cyclo- |
| phenyl)thiazole | propylmethanone oxime |
| 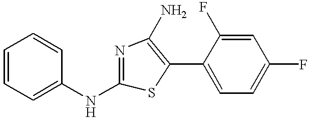 | 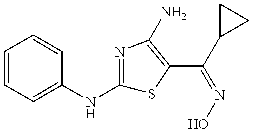 |
| N²-phenyl-5-pyridin-3-ylthiazole-2,4-diamine | N-[4-amino-5-(pyridine-3-carbonyl)thiazol-2- |
| | yl]benzamide |
| 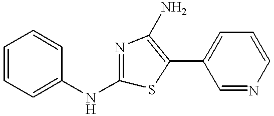 | 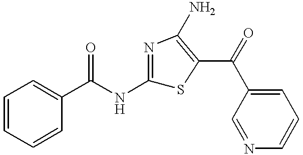 |
| (4-amino-2-benzylaminothiazol-5-yl)phenyl- | [4-amino-2-(pyridin-4-ylamino)thiazol-5-yl]- |
| methanone | phenylmethanone |
| 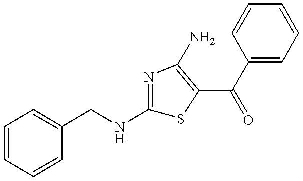 | 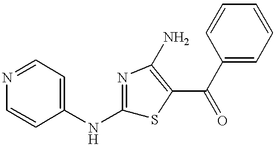 |
| 3-[4-amino-5-(pyridine-3-carbonyl)thiazol-2- | 3-[4-amino-5-(3-benzyloxybenzoyl)thiazol-2- |
| ylamino]propionic acid | ylamino]propionic acid |
| 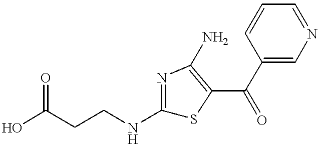 | 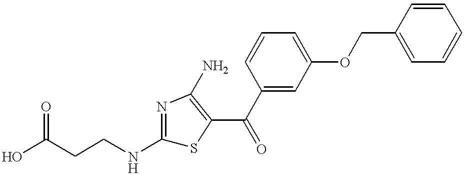 |
| (4-amino-2-propylaminothiazol-5-yl)-(3-benz- | (4-amino-2-propylaminothiazol-5-yl)-[3-(thia- |
| yloxyphenyl)methanone | zol-5-ylmethoxy)phenyl]methanone |
| 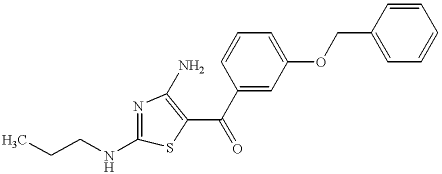 | 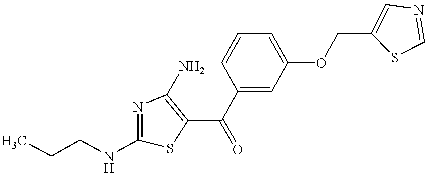 |
| (4-amino-2-phenylaminothiazol-5-yl)-[3-(3,4- | (4-amino-2-phenylaminothiazol-5-yl)-(3,4-di- |
| dichlorobenzyloxy)phenyl)methanone | chlorobiphenyl-4-yl)methanone |
| 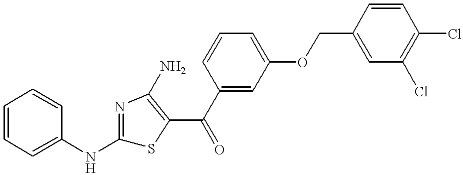 | 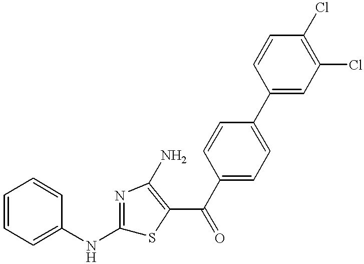 |
| (4-amino-2-phenylaminothiazol-5-yl)-(4′-tri- | 4-amino-2-phenylaminothiazole-5-carboxylic |
| fluorobiphenyl-4-yl)methanone | acid (4-trifluoromethylphenyl)-amide |
| 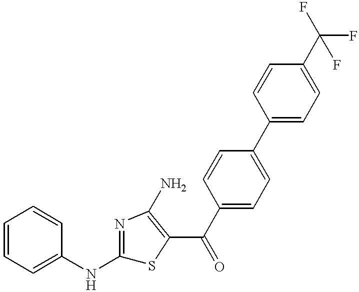 | 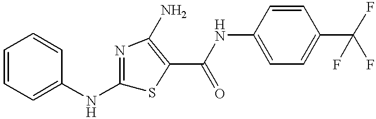 |
| 4-amino-2-phenylaminothiazole-5-carboxylic | 4-amino-2-phenylaminothiazole-5-carboxylic |
| acid (4-tert-butylphenyl)amide | acid (4-phenoxyphenyl)amide |
| 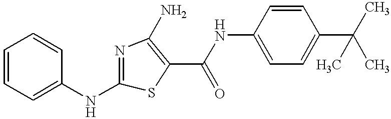 | 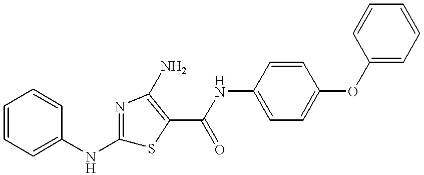 |
| N-[4-amino-5-(5-chlorothiphene-2-carbonyl)- | |
| thiazol-2-yl]acetamide | |
| 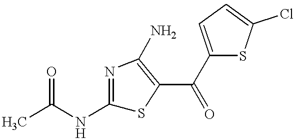 | |

ASSAY I

Inhibition of GSK-3 by a test compound was evaluated using human GSK-3β and a glycogen synthase derived substrate with the following amino acid sequence:

YRRAAVPPSPSLSRHSSPHQS(PO₄)EDEEE-NH₂.

In brief, GSK-3β was incubated with 32 μM substrate and varying concentrations of test compound in a buffer containing 0.1 mM ³³P-labeled ATP, 10 mM magnesium acetate, 8 mM MOPS pH 7.0, 0.2 mM EDTA, 0.1% dithiothreitol and 0.03% Triton-X100 for 60 min at room temperature. The reaction was performed using 96-well filter plates. The reaction was terminated by filtration followed by addition of 25 μl 2% phosphoric acid to each well. All wells were then washed three times in 0.5% phosphoric acid to remove unreacted ³³P-labeled ATP, dried and radioactivity was counted in a Packard topcounter. Dose-response profiles were generated, and the IC₅₀ value for inhibition of GSK-3 by the test compound was calculated using a four-parameter logistic function.

The following compounds inhibited GSK-3 with an IC₅₀ value lower than 5 μM: examples 1, 2, 4, 5, 11-29, 32, 34, 36, 39, 41, 42, 44, 47, 50, 52, 53, 54 and 58.

ASSAY II

The insulin releasing effect of the compounds were evaluated by the following test method:

Preparation of islets and single B-cells

Pancreatic islets were isolated from the pancreas from fed NMRI mice (20-25 g) by collagenase digestion. For insulin release experiments, the islets were kept in RPMI-1640 tissue culture medium (Gibco) overnight before use. Alternatively, the islets were dispersed into single cells by shaking in a Ca²⁺-free solution and the resulting cell suspension was plated on Nunc petri dishes and maintained for up to 3 days in RPMI-1640 medium supplemented with 10% heat-inactivated fetal calf serum, 100 i.u./ml penicilin and 100 μg/ml streptomycin. The cells were plated in 24-well plates 2 days before initiation of the experiment.

Electrophysiology K_{ATP}

Patch pipettes were pulled from borosilicate glass capillaries, coated with Sylgard at their tips and fire-polished before use. The pipette resistance (when filled with the pipette-filling solutions) was 2-4 MΩ. All currents have been filtered at 1 kHz using the internal filters of the amplifiers and acquired at a rate of 3 kHz. The zero-current potential was adjusted before establishment of the seal with the pipette in the bath. The whole-cell KATP conductance was estimated by applying 10 mV hyper- and depolarizing voltage pulses (duration: 200 ms; pulse interval: 2 s) from a holding potential of −70 mV using the standard whole-cell configuration of the patch-clamp technique. The currents were recorded using an Axopatch 200B patch clamp amplifier (Axon Instruments, Foster City, Calif., USA), digitized and stored in a computer using the Digidata AD-converter and the software pClamp (version 6.0; Axon Instruments).

Solutions

The extracellular medium consisted of (in mM) 138 NaCl, 5.6 KCl, 2.6 CaCl₂, 1.2 MgCl₂, 5 HEPES (pH 7.4 with NaOH) and 5 D-glucose. The volume of the recording chamber was approximately 0.4 ml and the solution entering the bath (1.5-2 ml/min) was maintained at 33° C. for measurements of exocytosis. For recordings of whole-cell K_{ATP}-channel activity, the pipette solution contained (in mM) 125 KCl, 30 KOH, 10 EGTA, 1 MgCl₂, 5 HEPES, 0.3 Mg-ATP and 0.3 K-ADP (pH 7.15 with KOH). The electrode solution for measurements of exocytosis consisted of (in mM) 125 K-glutamate, 10 KCl, 10 NaCl, 1 MgCl₂, 5 HEPES, 3 Mg-ATP, 10 EGTA, 5 CaCl₂. The free Ca²⁺ concentration of the resulting buffer was 0.22 μM.

Insulin release

Intact pancreatic islets were isolated from fed female NMRI mice (15-18 g) as previously described (Fuhlendorff et al, Diabetes, Vol. 47 (3) pp. 345-351 (1998)). Insulin release was measured from groups of 10 size-matched islets, cultured overnight in RPMI-1640 tissue culture medium containing 1% glutamax, 1% penicillin/streptomycin, 7.5% NaHCO₃ and 10% normal calf serum. The islets were washed for approx. 20 min in Krebs Ringer Buffer (0.115 M NaCl, 0.0047 M KCl, 0.0026 M CaCl₂, 0.0012 M KH₂PO₄, 0.0012 M MgSO₄, 1M HEPES buffer, 2 mM glutamin, 5 mM NaHCO₂, 0.2% human serum albumin and 1% penicillinistreptomycin) containing the same glucose concentration (2.5-10 mM) as the later incubation with the test compound. After washing the islets were incubated for 1 hour at 37° C. in Krebs Ringer Buffer containing the test compound at 100 μM and glucose at 2.5-10 mM. After the 1 hour incubation the medium was aspirated and kept at −20° C. until assayed for insulin using an ELISA technique.

ELISA

96-well NUNC-immuno plates (MaxiSorP) were coated over night at 4° C. with a rabbit-anti-guinea pig-lgG (Dako) antibody diluted 1:1000 in 0.1 M NaHCO₃ pH 9.8. After washing 4 times in 0.15 M NaCl and 0.005% Tween-20 the plates were incubated with guinea pig-anti-insulin diluted in phosphate buffered saline (pH 7.4) containing 0.1% Tween-20 and 0.5% human serum albumin (PBS-DIL) over night. After washing the samples are then incubated for 1.5 hours with relevant samples and porcine insulin (Sigma) diluted 1:10000 in PBS-DIL. The samples are then washed and developed with 3,3′, 5,5═-tetramethylbenzidine substrate as prescribed by the manufacturer (KEM EN TEC). The amount of insulin is quantified from a standard curve after reading the samples at 450 nm.

From the foregoing, it will be appreciated that, although specific embodiments of the invention have been described herein for the purposes of illustration, various modifications may be made without deviating from the spirit and scope of the invention as defined by the appended claims.

1. Use of a compound of the formula (I): 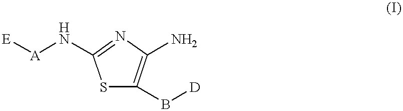
wherein
E is C₁₋₆-alkyl, C₂₋₆-alkenyl, C₂₋₆-alkynyl, C₁₋₆-alkylthio, C₁₋₆-alkoxy, C₁₋₆-alkanoyloxy, —C(═O)OH, —C(═O)O—C₁₋₆-alkyl, or 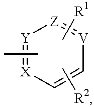
X, Y, Z and V independently are ═CH— or ═N—, with the proviso that at least two of X, Y, Z and V are ═CH—,
R¹ and R² which may be the same or different independently are selected from
hydrogen, hydroxy, halogen, cyano, nitro, —NR³R⁴, —C(═O)NR³R⁴, —OC(═O)NR³R⁴, —OCH₂C(═O)NR³R⁴, C₁₋₆-alkoxy, —C(═O)OR³, —C(═O)R³, —NHC(═O)R³, —CHF₂, —CF₃, —OCF₃, —OCHF₂, —OCH₂CF₃, —OCF₂CHF₂, —SCF₃, —SR³, —S(═O)R³, —S(═O)₂R³, —S(═O)₂NH₂,
wherein R³ and R⁴ which may be the same or different independently are selected from hydrogen and C₁₋₆-alkyl or R³ and R⁴ together with the nitrogen atom to which they are attached form a 3 to 8 membered cyclic ring optionally containing one or two further heteroatoms selected from oxygen, sulfur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR⁵R⁶, —C(═O)NR⁵R⁶, —OC(═O)NR⁵R⁶, —OCH₂C(═O)NR⁵R⁶, C₁₋₆-alkoxy, —C(═O)OR⁵, —C(O)R⁵, —NHC(═O)R⁵, —CHF₂, —CF₃, —OCF₃, —OCHF₂, —OCH₂CF₃, —OCF₂CHF₂, —SCF₃, —SR⁵, —S(═O)R⁵, —S(═O)₂R⁵, —S(═O)₂NH₂,
wherein R⁵ and R⁶ which may be the same or different independently are selected from hydrogen and C₁₋₆-alkyl or R⁵ and R⁶ together with the nitrogen atom to which they are attached form a 3 to 8 membered cyclic ring optionally containing one or two further heteroatoms selected from oxygen, sulfur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkyl-carbonyl, heteroaroyl, C₃₋₈-heterocyclylcarbonyl, —O-aryl, —O—C₃₋₈-cycloalkyl, —O-heteroaryl, —O—C₃₋₈-heterocyclyl, —S-aryl, —C₃₋₈-cycloalkyl, —S-heteroaryl, —S—C₃₋₈-heterocyclyl,
which may optionally be substituted with one to three substituents selected from
hydroxy, halogen, cyano, nitro, —NR⁷R⁸, —C(═O)NR⁷R⁸, —OC(═O)NR⁷R⁸, —OCH₂C(═O)NR⁷R⁸, C₁₋₆-alkoxy, —C(═O)OR⁷, —C(═O)R⁷, —NHC(═O)R⁷, —CHF₂, —CF₃, —OCF₃, —OCHF₂, —OCH₂CF₃, —OCF₂CHF₂, —SCF₃, —SR⁷, —S(═O)R⁷, —S(═O)₂R⁷, —S(═O)₂NH₂,
wherein R⁷ and R⁸ which may be the same or different independently are selected from hydrogen and C₁₋₆-alkyl or R⁷ and R⁸ together with the nitrogen atom to which they are attached form a 3 to 8 membered cyclic ring optionally containing one or two further heteroatoms selected from oxygen, sulfur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR⁹R¹⁰, —C(═O)NR⁹R¹⁰, —OC(═O)NR⁹R¹⁰, —OCH₂C(═O)NR⁹R¹⁰, C₁₋₆-alkoxy, —C(═O)OR⁹, —C(═O)R⁹, —NHC(═O)R⁹, —CHF₂, —CF₃, —OCF₃, —OCHF₂, —OCH₂CF₃, —OCF₂CHF₂, —SCF₃, —SR⁹, —S(═O)R⁹, —S(═O)₂R⁹, —S(═O)₂NH₂,
wherein R⁹ and R¹⁰ which may be the same or different independently are selected from hydrogen and C₁₋₆-alkyl or R⁹ and R¹⁰ together with the nitrogen atom to which they are attached form a 3 to 8 membered cyclic ring optionally containing one or two further heteroatoms selected from oxygen, sulfur and nitrogen,
A is a valence bond, C₁₋₆-alkylene or —C(═O)—,
B is a valence bond, —C(═O)—, —S(═O)—, —S(═O)₂— or —C(═N-OR¹¹)—,
R¹¹ is hydrogen, C₁₋₆-alkyl or aryl-C₁₋₆-alkyl,
D is
hydroxy, halogen, cyano, nitro, —NR¹²R¹³, —N(R¹²)OR¹³, —C(═O)NR¹²R¹³, —OC(═O)NR¹²R¹³, —OCH₂C(═O)NR¹²R¹³, C₁₋₆-alkoxy, —C(═O)OR¹², —C(═O)R¹², —NHC(═O)R¹², —CHF₂, —CF₃, —OCF₃, —OCHF₂, —OCH₂CF₃, —OCF₂CHF₂, —SCF₃, —SR¹², —S(═O)R¹², —S(═O)₂R¹², —S(═O)₂NH₂,
wherein R¹² and R¹³ which may be the same or different independently are selected from hydrogen and C₁₋₆-alkyl or R¹² and R¹³ together with the nitrogen atom to which they are attached form a 3 to 8 membered cyclic ring optionally containing one or two further heteroatoms selected from oxygen, sulfur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR¹⁴R¹⁵, —C(═O)NR¹⁴R¹⁵, —OC(═O)NR¹⁴R¹⁵, —OCH₂C(═O)NR¹⁴R¹⁵, C₁₋₆-alkoxy, —C(═O)OR¹⁴, —C(═O)R¹⁴, —NHC(═O)R¹⁴, —CHF₂, —CF₃, —OCF₃, —OCHF₂, —OCH₂CF₃, —OCF₂CHF₂, —SCF₃, —SR¹⁴, —S(═O)R¹⁴, —S(═O)₂R¹⁴, —S(═O)₂NH₂,
wherein R¹⁴ and R¹⁵ which may be the same or different independently are selected from hydrogen and C₁₋₆-alkyl or R¹⁴ and R¹⁵ together with the nitrogen atom to which they are attached form a 3 to 8 membered cyclic ring optionally containing one or two further heteroatoms selected from oxygen, sulfur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkyl-carbonyl, heteroaroyl, C₃₋₈-heterocyclylcarbonyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl, —NH-aryl, —NH-heteroaryl,
which may optionally be substituted with one to three substituents selected from
hydroxy, halogen, cyano, nitro, —NR¹⁶R¹⁷, —C(═O)NR¹⁶R¹⁷, —OC(═O)NR¹⁶R¹⁷, —OCH₂C(═O)NR¹⁶R¹⁷, C₁₋₆-alkoxy, —C(═O)OR¹⁶, —C(═O)R¹⁶, —NHC(═O)R¹⁶, —CHF₂, —CF₃, —OCF₃, —OCHF₂, —OCH₂CF₃, —OCF₂CHF₂, —SCF₃, —SR¹⁶, —S(═O)R¹⁶, —S(═O)₂R¹⁶, —S(═O)₂NH₂,
wherein R¹⁶ and R¹⁷ which may be the same or different independently are selected from hydrogen and C₁₋₆-alkyl or R¹⁶ and R¹⁷ together with the nitrogen atom to which they are attached form a 3 to 8 membered cyclic ring optionally containing one or two further heteroatoms selected from oxygen, sulfur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR¹⁸R¹⁹, —C(═O)NR¹⁸R¹⁹, —OC(═O)NR¹⁸R¹⁹, —OCH₂C(═O)NR¹⁸R¹⁹, C₁₋₆-alkoxy, —C(═O)OR¹⁸, —C(═O)R¹⁸, —NHC(═O)R¹⁸, —CHF₂, —CF₃, —OCF₃, —OCHF₂, —OCH₂CF₃, —OCF₂CHF₂, —SCF₃, —SR¹⁸, —S(═O)R¹⁸, —S(═O)₂R¹⁸, —S(═O)₂N H₂,
wherein R¹⁸ and R¹⁹ which may be the same or different independently are selected from hydrogen and C₁₋₆-alkyl or R¹⁸ and R¹⁹ together with the nitrogen atom to which they are attached form a 3 to 8 membered cyclic ring optionally containing one or two further heteroatoms selected from oxygen, sulfur and nitrogen,
aryl, C₃₋₈-cycloalkyl, heteroaryl, C₃₋₈-heterocyclyl, aryl-C₁₋₆alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkyl-carbonyl, heteroaroyl, C₃₋₈-heterocyclylcarbonyl, —O-aryl, —O—C₃₋₈-cycloalkyl, —O-heteroaryl, —O—C₃₋₈-heterocyclyl, —S-aryl, —S—C₃₋₈-cycloalkyl, —S-heteroaryl, —S—C₃₋₈-heterocyclyl,
which may optionally be substituted with one to three substituents selected from hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆-alkyl, C₂₋₆-alkenyl, C₂₋₆-alkynyl, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰ —S(═O)₂R²¹, —S(═O)₂NH₂,
wherein R²⁰ and R²¹ which may be the same or different independently are selected from hydrogen and C₁₋₆ alkyl or R²⁰ and R²¹ together with the nitrogen atom to which they are attached form a 3 to 8 membered cyclic ring optionally containing one or two further heteroatoms selected from oxygen, sulfur and nitrogen,
as well as any optical or geometric isomer or tautomeric form thereof including mixtures of these or a pharmaceutically acceptable salt thereof for the preparation of a pharmaceutical composition for the treatment and/or prevention of diseases and disorders wherein an inhibition of GSK-3 is beneficial.

2. Use of a compound according to claim 1 wherein A is a valence bond.

3. Use of a compound according to claim 1 wherein A is C₁₋₆ alkylene.

4. Use of a compound according to any one of the claims 1 to 3 wherein E is C₁₋₆ alkyl, C₂₋₆-alkoxy or —C(O)O—C₁₋₆ alkyl.

5. Use of a compound according to any one of the claims 1 to 3 wherein E is 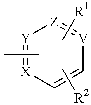
wherein X, Y, Z, V, R¹ and R² are as defined in claim 1.

6. Use of a compound according to claim 5 wherein X, Y, Z and V are all ═CH—.

7. Use of a compound according to claim 5 or 6 wherein R¹ and R² which may be the same or different independently are selected from
hydrogen, hydroxy, halogen, cyano, nitro, —NR³R⁴, —C(═O)NR³R⁴, —OC(═O)NR³R⁴, OCH₂C(═O)NR³R⁴, C₁₋₆-alkoxy, —C(═O)OR³, —C(═O)R³, —NHC(═O)R³, —CHF₂, —CF₃, —OCF₃, —OCHF₂, —OCH₂CF₃, —OCF₂CHF₂, —SCF₃, —SR³, —S(═O)R³, —S(═O)₂R³, —S(═O)₂NH₂,
wherein R³ and R⁴ which may be the same or different independently are selected from hydrogen and C₁₋₆-alkyl or R³ and R⁴ together with the nitrogen atom to which they are attached form a 3 to 8 membered cyclic ring optionally containing one or two further heteroatoms selected from oxygen, sulfur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR⁵R⁶, —C(═O)NR⁵R⁶, —OC(═O)NR⁵R⁶, —OCH₂C(═O)NR⁵R⁶, C₁₋₆-alkoxy, —C(═O)OR⁵, —C(═O)R⁵, —NHC(═O)R⁵, —CHF₂, —CF₃, —OCF₃, —OCHF₂, —OCH₂CF₃, —OCF₂CHF₂, —SCF₃, —SR⁵, —S(═O)R⁵, —S(═O)₂R⁵, —S(═O)₂NH₂,
wherein R⁵ and R⁶ which may be the same or different independently are selected from hydrogen and C₁₋₆-alkyl or R⁵ and R⁶ together with the nitrogen atom to which they are attached form a 3 to 8 membered cyclic ring optionally containing one or two further heteroatoms selected from oxygen, sulfur and nitrogen.

8. Use of a compound according to claim 7 wherein R¹ and R² which may be the same or different independently are selected from hydrogen or halogen.

9. Use of a compound according to claim 8 wherein R¹ and R² are both hydrogen.

10. Use of a compound according to any one of the preceding claims wherein B is —C(═O)—.

11. Use of a compound according to any one of the preceding claims wherein B is a valence bond.

12. Use of a compound according to any one of the preceding claims wherein D is
hydroxy, —NR¹²R¹³, —C(O)R¹², cyano, —N(R¹²)OR¹³, C₁₋₆-alkoxy, —C(═O)OR¹², —CF₃, C₁₋₆-alkyl,
wherein R¹² and R¹³ which may be the same or different independently are selected from hydrogen and C₁₋₆-alkyl or R¹² and R¹³ together with the nitrogen atom to which they are attached form a 3 to 8 membered cyclic ring optionally containing one or two further heteroatoms selected from oxygen, sulfur and nitrogen,
aryl, C₃₋₈-cycloalkyl, heteroaryl, C₃₋₈-heterocyclyl, aryl-C₁₋₆-alkoxy, —NH-aryl,
which may optionally be substituted with one to three substituents selected from
hydroxy, halogen, cyano, nitro, —NR¹⁶R¹⁷, —C(═O)NR¹⁶R¹⁷, —OC(═O)NR¹⁶R¹⁷, —OCH₂C(═O)NR¹⁶R¹⁷, C₁₋₆-alkoxy, —C(═O)OR¹⁶, —C(═O)R¹⁶, —NHC(═O)R¹⁶, —CHF₂, —CF₃, —OCF₃, —OCHF₂, —OCH₂CF₃, —OCF₂CHF₂, —SCF₃, —SR¹³, —S(═O)R¹⁶, —S(═O)₂R¹⁶, —S(═O)₂NH₂,
wherein R¹⁶ and R¹⁷ which may be the same or different independently are selected from hydrogen and C₁₋₆-alkyl or R¹⁶ and R¹⁷ together with the nitrogen atom to which they are attached form a 3 to 8 membered cyclic ring optionally containing one or two further heteroatoms selected from oxygen, sulfur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR¹⁸R¹⁹, —C(═O)NR¹⁸R¹⁹, —OC(═O)NR¹⁸R¹⁹, —OCH₂C(═O)NR¹⁸R¹⁹, C₁₋₆-alkoxy, —C(═O)OR¹⁸, —C(═O)R¹⁸, —NHC(═O)R¹⁸, —CHF₂, —CF₃, —OCF₃, —OCHF₂, —OCH₂CF₃, —OCF₂CHF₂, —SCF₃, —SR¹⁸, —S(═O)R¹⁸, —S(═O)₂R¹⁸, —S(═O)₂NH₂,
wherein R¹⁸ and R¹⁹ which may be the same or different independently are selected from hydrogen and C₁₋₆-alkyl or R¹⁸ and R¹⁹ together with the nitrogen atom to which they are attached form a 3 to 8 membered cyclic ring optionally containing one or two further heteroatoms selected from oxygen, sulfur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkyl-carbonyl, heteroaroyl, C₃₋₈-heterocyclylcarbonyl, —O-aryl, —O—C₃₋₈-cycloalkyl, —O—heteroaryl, —O—C₃₋₈-heterocyclyl, —S-aryl, —S—C₃₋₈-cycloalkyl, —S-heteroaryl, —S—C₃₋₈-heterocyclyl,
which may optionally be substituted with one to three substituents selected from hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆-alkyl, C₂₋₆-alkenyl, C₂₋₆-alkynyl, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl or R²⁰ and R²¹ together with the nitrogen atom to which they are attached form a 3 to 8 membered cyclic ring optionally containing one or two further heteroatoms selected from oxygen, sulfur and nitrogen.

13. Use of a compound according to claim 12 wherein D is C₃₋₈-cycloalkyl, aryl or heteroaryl, which are optionally substituted with one or more substituents selected from halogen, C₁₋₆-alkyl, and phenyl-C₁₋₆-alkoxy.

14. Use of a compound according to claim 12 wherein D is
hydroxy, —NR¹²R¹³, C₁₋₆-alkoxy, —C(═O)OR¹², —CF₃, C₁₋₆-alkyl,
wherein R¹² and R¹³ which may be the same or different independently are selected from hydrogen and C₁₋₆ alkyl or R¹² and R¹³ together with the nitrogen atom to which they are attached form a 3 to 8 membered cyclic ring optionally containing one or two further heteroatoms selected from oxygen, sulfur and nitrogen,
aryl, C₃₋₈-cycloalkyl, heteroaryl, C₃₋₈-heterocyclyl, aryl-C₁₋₆-alkoxy,
which may optionally be substituted with one to three substituents selected from
hydroxy, halogen, cyano, nitro, —NR¹⁶R¹⁷, —C(═O)NR¹⁶R¹⁷, —OC(═O)NR¹⁶R¹⁷, —OCH₂C(═O)NR¹⁶R¹⁷, C₁₋₆-alkoxy, —C(═O)OR¹⁶, —C(═O)R¹⁶, —NHC(═O)R¹⁶, —CHF₂, —CF₃, —OCF₃, —OCHF₂, —OCH₂CF₃, —OCF₂CHF₂, —SCF₃, —SR¹⁶, —S(═O)R¹⁶, —S(═O)₂R¹⁶, —S(═O)₂NH₂,
wherein R¹⁶ and R¹⁷ which may be the same or different independently are selected from hydrogen and C₁₋₆-alkyl or R¹⁶ and R¹⁷ together with the nitrogen atom to which they are attached form a 3 to 8 membered cyclic ring optionally containing one or two further heteroatoms selected from oxygen, sulfur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR¹⁸R¹⁹, —C(═O)NR¹⁸R¹⁹, —OC(═O)NR¹⁸R¹⁹, —OCH₂C(═O)NR¹⁸R¹⁹, C₁₋₆-alkoxy, —C(═O)OR¹⁸, —C(═O)R¹⁸, —NHC(═O)R¹⁸, —CHF₂, —CF₃, —OCF₃, —OCHF₂, —OCH₂CF₃, —OCF₂CHF₂, —SCF₃, —SR¹⁸, —S(═O)R¹⁸, —S(═O)₂R¹⁸, —S(═O)₂NH₂,
wherein R¹⁸ and R¹⁹ which may be the same or different independently are selected from hydrogen and C₁₋₆-alkyl or R¹⁸ and R¹⁹ together with the nitrogen atom to which they are attached form a 3 to 8 membered cyclic ring optionally containing one or two further heteroatoms selected from oxygen, sulfur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cyclo-alkyl-C₁₋₆-alkoxy, heteroaryl-C₁₋₆-alkoxy, C₃₋₈-heterocyclyl-C₁₋₆-alkoxy, aroyl, C₃₋₈-cyclo-alkylcarbonyl, heteroaroyl, C₃₋₈-heterocyclylcarbonyl, —O-aryl, —O—C₃₋₈-cycloalkyl, —O—heteroaryl, —O—C₃₋₈-heterocyclyl, —S-aryl, —S—C₃₋₈-cycloalkyl, —S-heteroaryl, —S—C₃₋₈-heterocyclyl,
which may optionally be substituted with one to three substituents selected from hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆-alkyl, C₂₋₆-alkenyl, C₂₋₆-alkynyl, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl or R²⁰ and R²¹ together with the nitrogen atom to which they are attached form a 3 to 8 membered cyclic ring optionally containing one or two further heteroatoms selected from oxygen, sulfur and nitrogen.

15. Use of the compounds according to claim 14 wherein D is
hydroxy, —NR¹²R¹³, C₁₋₆-alkoxy, —C(═O)OR¹², —CF₃, C₁₋₆-alkyl,
wherein R¹² and R¹³ which may be the same or different independently are selected from hydrogen and C₁₋₆-alkyl,
cyclopropyl, phenyl, naphthyl, morpholino, pyridinyl, tetrahydropyridinyl, thiophenyl, benzothiophenyl, phenyl-C₁₋₆-alkoxy,
which may optionally be substituted with one to three substituents selected from
hydroxy, halogen, nitro, —NR¹⁶R¹⁷, C₁₋₆-alkoxy, —CF₃, —OCF₃, C₁₋₆-alkyl,
wherein R¹⁶ and R¹⁷ which may be the same or different independently are selected from hydrogen and C₁₋₆-alkyl,
phenyl, phenyl-C₁₋₆-alkoxy,
which may optionally be substituted with one to three substituents selected from halogen, nitro, —NR²⁰R²¹, C₁₋₆-alkoxy, —CF₃, —OCF₃, C₁₋₆-alkyl,
wherein R²⁰ and R²¹ which may be the same or different independently are selected from hydrogen and C₁₋₆-alkyl.

16. Use of the compounds according to claim 15 wherein D is
hydroxy, —NR¹²R¹³, C₁₋₆-alkoxy, —C(═O)OR¹², —CF₃, C₁₋₆-alkyl,
wherein R¹² and R¹³ which may be the same or different independently are selected from hydrogen and C₁₋₆-alkyl,
cyclopropyl, phenyl, naphthyl, morpholino, pyridinyl, thiophenyl, benzothiophenyl, phenyl-C₁₋₆-alkoxy,
which may optionally be substituted with one to three substituents selected from
hydroxy, halogen, nitro, —NR¹⁶R¹⁷, C₁₋₆-alkoxy, —CF₃, —OCF₃, C₁₋₆-alkyl,
wherein R¹⁶ and R¹⁷ which may be the same or different independently are selected from hydrogen and C₁₋₆alkyl,
phenyl, phenyl-C₁₋₆-alkoxy,
which may optionally be substituted with one to three substituents selected from halogen, nitro, —NR²⁰R²¹, C₁₋₆-alkoxy, —CF₃, —OCF₃, C₁₋₆-alkyl,
wherein R²⁰ and R²¹ which may be the same or different independently are selected from hydrogen and C₁₋₆-alkyl.

17. Use of the compounds according to claim 15 wherein D is
cyclopropyl, phenyl, pyridinyl, tetrahydropyridinyl, thiophenyl,
which may optionally be substituted with one to three substituents selected from
hydroxy, halogen, nitro, —NR¹⁶R¹⁷, C₁₋₆-alkoxy, —CF₃, —OCF₃, C₁₋₆-alkyl,
wherein R¹⁶ and R¹⁷ which may be the same or different independently are selected from hydrogen and C₁₋₆-alkyl,
phenyl, phenyl-C₁₋₆-alkoxy,
which may optionally be substituted with one to three substituents selected from halogen, nitro, —NR²⁰R²¹, C₁₋₆-alkoxy, —CF₃, —OCF₃, C₁₋₆-alkyl,
wherein R²⁰ and R²¹ which may be the same or different independently are selected from hydrogen and C₁₋₆-alkyl.

18. Use of the compounds according to claim 17 wherein D is cyclopropyl, phenyl, pyridinyl, tetrahydropyridinyl or thiophenyl, which are optionally substituted with one to three substituents selected from halogen, C₁₋₆-alkyl and phenyl-C₁₋₆-alkoxy.

19. Use of the compounds according to claim 18 wherein D is cyclopropyl or pyridinyl.

20. Use of a compound according to any one of the claims 1 to 19 for the preparation of a pharmaceutical composition for the treatment and/or prevention of diseases and disorders related to GSK-3.

21. Use of a compound according to any one of the claims 1 to 19 for the preparation of a pharmaceutical composition for the treatment and/or prevention of diseases and disorders wherein growth factor induced inhibition of GSK-3 is insufficient.

22. Use of a compound according to any one of the claims 1 to 19 for the preparation of a pharmaceutical composition for the treatment and/or prevention of diseases and disorders wherein glycogen metabolism exhibits abnormalities.

23. Use of a compound according to any one of the claims 1 to 19 for the preparation of a pharmaceutical composition for the treatment and/or prevention of diseases and disorders wherein glycogen synthase is insufficiently activated.

24. Use of a compound according to any one of the claims 1 to 19 for the preparation of a pharmaceutical composition for the treatment and/or prevention of diseases and disorders involving elevated blood glucose.

25. Use of a compound according to any one of the claims 1 to 19 for the preparation of a pharmaceutical composition for the treatment and/or prevention of hyperglycemia.

26. Use of a compound according to any one of the claims 1 to 19 for the preparation of a pharmaceutical composition for the treatment and/or prevention of IGT.

27. Use of a compound according to any one of the claims 1 to 19 for the preparation of a pharmaceutical composition for the treatment and/or prevention of Type 2 diabetes.

28. Use of a compound according to any one of the claims 1 to 19 for the preparation of a pharmaceutical composition for the treatment and/or prevention of Type 1 diabetes.

29. Use of a compound according to any one of the claims 1 to 19 for the preparation of a pharmaceutical composition for the treatment and/or prevention of obesity.

30. Use of a compound according to any one of the claims 1 to 19 for the preparation of a pharmaceutical composition for the treatment and/or prevention of Alzheimer's disease.

31. Use of a compound according to any one of the claims 1 to 19 for the preparation of a pharmaceutical composition for the treatment and/or prevention of bipolar disorder.

32. Use according to any one of the claims 20 to 31 in combination with one or more further active agents selected from antidiabetic compounds, antihyperlipidemic compounds, antiobesity compounds and antihypertensive compounds.

33. Use according to claim 30 or 31 in combination with one or more further active agents selected from agents for the treatment and/or prevention of Alzheimer's disease and agents for the treatment and/or prevention of bipolar disease.

34. A compound of the general formula (Ia): 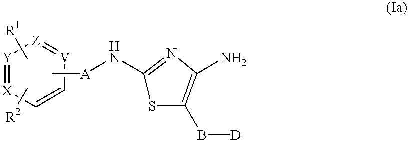
wherein
A is C₁₋₆-alkylene or —C(═O)—,
R¹, R², X, Y. Z. V, B and D are as defined in claim 1, with the provisos that when
X, Y, Z and V are all ═CH—, A is ethylene, B is —C(═O)—, R¹ is hydrogen, D is 2-nitrophenyl, R² must not be hydrogen or 4-chloro,
X, Y, Z and V are all ═CH—, A is ethylene, B is —C(═O)—, R¹ is hydrogen, D is 2-methoxy-phenyl, R² must not be 4-chloro,
X, Y, Z and V are all ═CH—, A is methylene, B is —C(═O)—, R¹ is hydrogen, R² is 4-methoxy, D must not be 5-chlorobenzofuran-2-yl or 2,5-dimethylthiophen-3-yl,
X, Y, Z and V are all ═CH—, A is methylene, B is —C(═O)—, R¹ and R² are both hydrogen, D must not be methyl,
as well as any optical or geometric isomer or tautomeric form thereof including mixtures of these or a pharmaceutically acceptable salt thereof.

35. A compound of the general formula (lb): 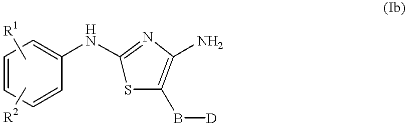
wherein
D is
hydroxy, halogen, cyano, nitro, —NR¹²R¹³, —N(R¹²)OR¹³, —C(═O)NR¹²R¹³, —OC(═O)NR¹²R¹³, —OCH₂C(═O)NR¹²R¹³, C₁₋₆-alkoxy, —C(═O)OR¹², —C(═O)R¹², —NHC(═O)R¹², —CHF₂, —CF₃, —OCF₃, —OCHF₂, —OCH₂CF₃, —OCF₂CHF₂, —SCF₃, —SR¹², —S(═O)R¹², —S(═O)₂R¹², —S(═O)₂NH₂,
wherein R¹² and R¹³ which may be the same or different independently are selected from hydrogen and C₁₋₆-alkyl or R¹² and R¹³ together with the nitrogen atom to which they are attached form a 3 to 8 membered cyclic ring optionally containing one or two further heteroatoms selected from oxygen, sulfur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR¹⁴R¹⁵, —C(═O)NR¹⁴R¹⁵, —OC(═O)NR¹⁴R¹⁵, —OCH₂C(═O)NR¹⁴R¹⁵, C₁₋₆-alkoxy, —C(═O)OR¹⁴, —C(═O)R¹⁴, —NHC(═O)R¹⁴, —CHF₂, —CF₃, —OCF₃, —OCHF₂, —OCH₂CF₃, —OCF₂CHF₂, —SCF₃, —SR¹⁴, —S(═O)R¹⁴, —S(═O)₂R¹⁴, —S(═,)₂NH₂,
wherein R¹⁴ and R¹⁵ which may be the same or different independently are selected from hydrogen and C₁₋₆-alkyl or R¹⁴ and R¹⁵ together with the nitrogen atom to which they are attached form a 3 to 8 membered cyclic ring optionally containing one or two further heteroatoms selected from oxygen, sulfur and nitrogen,
C₃₋₈-cycloalkyl, aryl-C₁₋₆-alkoxy, naphthyl, benzothiophenyl,
which may optionally be substituted with one to three substituents selected from
hydroxy, halogen, cyano, nitro, —NR¹⁶R¹⁷, —C(═O)NR¹⁶R¹⁷, —OC(═O)NR¹⁶R¹⁷, —OCH₂C(═O)NR¹⁶R¹⁷, C₁₋₆-alkoxy, —C(═O)OR¹⁶, —C(═O)R¹⁶, —NHC(═O)R¹⁶, —CHF₂, —CF₃, —OCF₃, —OCHF₂, —OCH₂CF₃, —OCF₂CHF₂, —SCF₃, —SR¹⁶, —S(═O)R¹⁶, —S(═O)₂R¹⁶, —S(═O)₂NH₂,
wherein R¹⁶ and R¹⁷ which may be the same or different independently are selected from hydrogen and C₁₋₆-alkyl or R¹⁶ and R¹⁷ together with the nitrogen atom to which they are attached form a 3 to 8 membered cyclic ring optionally containing one or two further heteroatoms selected from oxygen, sulfur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR¹⁸R¹⁹, —C(═O)NR¹⁸R¹⁹, —OC(═O)NR¹⁸R¹⁹, —OCH₂C(═O)NR¹⁸R¹⁹, C₁₋₆-alkoxy, —C(═O)OR¹⁸, —C(═O)R¹⁸, —NHC(═O)R¹⁸, —CHF₂, —CF₃, —OCF₃, —OCHF₂, —OCH₂CF₃, —OCF₂CHF₂, —SCF₃, —SR¹⁸, —S(═O)R¹⁸, —S(═O)₂R¹⁸, —S(═O)₂NH₂,
wherein R¹⁸ and R¹⁹ which may be the same or different independently are selected from hydrogen and C₁₋₆alkyl or R¹⁸ and R¹⁹ together with the nitrogen atom to which they are attached form a 3 to 8 membered cyclic ring optionally containing one or two further heteroatoms selected from oxygen, sulfur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkyl-carbonyl, heteroaroyl, C₃₋₈-heterocyclylcarbonyl, —O-aryl, —O—C₃₋₈-cycloalkyl, —O—heteroaryl, —O—C₃₋₈-heterocyclyl, —S-aryl, —S—C₃₋₈-cycloalkyl, —S-heteroaryl, —S—C₃₋₈-heterocyclyl,
which may optionally be substituted with one to three substituents selected from hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆-alkyl, C₂₋₆-alkenyl, C₂₋₆-alkynyl, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆-alkyl or R²⁰ and R²¹ together with the nitrogen atom to which they are attached form a 3 to 8 membered cyclic ring optionally containing one or two further heteroatoms selected from oxygen, sulfur and nitrogen,
R¹, R² and B are as defined in claim 1,
with the provisos that when
R¹ and R² are both hydrogen, B is —C(═O)—, D must not be methyl, ethyl, cyclopropyl, methoxy, ethoxy or amino,
R¹ is hydrogen, R² is 4-dimethylamino, B is —C(═O)—, D must not be ethoxy, tert-butoxy, benzyloxy, 3-methylbenzothiophen-2-yl or 3-methyl-6-chlorobenzothiophen-2-yl,
R¹ is hydrogen, R² is 4-aminosulfonyl, B is —C(═O)—, D must not be 3-methylbenzothiophen-2-yl,
R¹ is hydrogen, R² is 4-chloro, B is —C(═O)—, D must not be methyl,
R¹ is hydrogen, R² is 3-methoxycarbonyl, B is —C(═O)—, D must not be naphthyl,
R¹ and R² are both hydrogen, B is a valence bond, D must not be cyano,
as well as any optical or geometric isomer or tautomeric form thereof including mixtures of these or a pharmaceutically acceptable salt thereof.

36. A compound of the general formula (Ic): 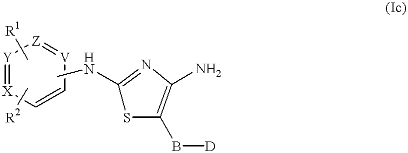
wherein one or two of X, Y, V and Z are ═N—, the rest being ═CH—,
R¹, R², B and D are as defined in claim 1,
with the provisos that when
R¹ and R² are hydrogen, B is —C(═O)—, X, Y and V are ═CH—, and Z is ═N— in the 3-position, D must not be phenyl, 2-nitrophenyl, 2,4-dimethylphenyl, 2,4-dichlorophenyl, 2-methoxyphenyl or naphthyl,
as well as any optical or geometric isomer or tautomeric form thereof including mixtures of these or a pharmaceutically acceptable salt thereof.

37. A compound of the general formula (Id): 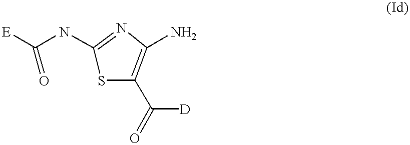
wherein E is C₁₋₆-alkyl, C₂₋₆-alkenyl, C₂₋₆-alkynyl, C₁₋₆-alkylthio, C₁₋₆-alkoxy, C₁₋₆-alkanoyloxy, —C(═O)OH or —C(═O)O—C₁₋₆-alkyl, and D is as defined in claim 1.

38. A compound according to claim 34 wherein A is C₁₋₆-alkylene.

39. A compound according to 34 wherein X, Y, Z and V are all ═CH—.

40. A compound according to any one of the claims 34 to 36 wherein B is —C(═O)—.

41. A compound according to any one of the claims 34 to 36 wherein B is a valence bond.

42. A compound according to any one of the claims 34 to 36 wherein R¹ and R² which may be the same or different independently are selected from
hydrogen, hydroxy, halogen, cyano, nitro, —NR³R⁴, —C(═O)NR³R⁴, —OC(═O)NR³R⁴, —OCH₂C(═O)NR³R⁴, C₁₋₆alkoxy, —C(═O)OR³, —C(═O)R³, —NHC(═O)R³, —CHF₂, —CF₃, —OCF₃, —OCHF₂, —OCH₂CF₃, —OCF₂CHF₂, —SCF₃, —SR³, —S(═O)R³, —S(═O)₂R³, —S(═O)₂NH₂,
wherein R³ and R⁴ which may be the same or different independently are selected from hydrogen and C₁₋₆-alkyl or R³ and R⁴ together with the nitrogen atom to which they are attached form a 3 to 8 membered cyclic ring optionally containing one or two further heteroatoms selected from oxygen, sulfur and nitrogen,
C₁₋₆-alkyl, C₂₋₆-alkenyI, C₂₋₆-alkynyl,
which may optionally be substituted with one or two substituents selected from hydroxy, halogen, cyano, nitro, —NR⁵R⁶, —C(═O)NR⁵R⁶, —OC(═O)NR⁵R⁶, —OCH₂C(═O)NR⁵R⁶, C₁₋₆-aIkoxy, —C(═O)OR⁵, —C(═O)R⁵, —NHC(═O)R⁵, —CHF₂, —CF₃, —OCF₃, —OCHF₂, —OCH₂CF₃, —OCF₂CHF₂, —SCF₃, —SR⁵, —S(═O)R⁵, —S(═O)₂R⁵, —S(═O)₂NH₂,
wherein R⁵ and R⁶ which may be the same or different independently are selected from hydrogen and C₁₋₆-alkyl or R⁵ and R⁶ together with the nitrogen atom to which they are attached form a 3 to 8 membered cyclic ring optionally containing one or two further heteroatoms selected from oxygen, sulfur and nitrogen.

43. A compound according to claim 42 wherein R¹ and R² which may be the same or different independently are selected from hydrogen or halogen.

44. A compound according to claim 43 wherein R¹ and R² are both hydrogen.

45. A compound according to any one of claims 34 or 36 to 44 wherein D is
hydroxy, —NR¹²R¹³, —C(O)R¹², cyano, —N(R¹²)OR¹³, C₁₋₆-alkoxy, —C(═O)OR¹², —CF₃, C₁₋₆-alkyl,
wherein R¹² and R¹³ which may be the same or different independently are selected from hydrogen and C₁₋₆-alkyl or R¹² and R¹³ together with the nitrogen atom to which they are attached form a 3 to 8 membered cyclic ring optionally containing one or two further heteroatoms selected from oxygen, sulfur and nitrogen,
aryl, C₃₋₈-cycloalkyl, heteroaryl, C₃₋₈-heterocyclyl, aryl-C₁₋₆-alkoxy, —NH-aryl,
which may optionally be substituted with one to three substituents selected from
hydroxy, halogen, cyano, nitro, —NR¹⁶R¹⁷, —C(═O)NR¹⁶R¹⁷, —OC(═O)NR¹⁶R¹⁷, —OCH₂C(═O)NR¹⁶R¹⁷, C₁₋₆-alkoxy, —C(═O)OR¹⁶, —C(═O)R¹⁶, —NHC(═O)R¹⁶, —CHF₂, —CF₃, —OCF₃, —OCHF₂, —OCH₂CF₃, —OCF₂CHF₂, —SCF₃, —SR¹⁶, —S(═O)R¹⁶, —S(═O)₂R¹⁶, —S(═O)₂NH₂,
wherein R¹⁶ and R¹⁷ which may be the same or different independently are selected from hydrogen and C₁₋₆-alkyl or R¹⁶ and R¹⁷ together with the nitrogen atom to which they are attached form a 3 to 8 membered cyclic ring optionally containing one or two further heteroatoms selected from oxygen, sulfur and nitrogen,
C₁₋₆-alkyl, C₂₋₆-alkenyl, C₂₋₆-alkynyl,
which may optionally be substituted with one or two substituents selected from hydroxy, halogen, cyano, nitro, —NR18R¹⁹, —C(═O)NR¹⁸R¹⁹, —OC(═O)NR¹⁸R¹⁹, —OCH₂C(═O)NR¹⁸R¹⁹, C₁₋₆-alkoxy, —C(═O)OR¹⁸, —C(═O)R¹⁸, —NHC(═O)R¹⁸, —CHF₂, —CF₃, —OCF₃, —OCHF₂, —OCH₂CF₃, —OCF₂CHF₂, —SCF₃, —SR¹⁸, —S(═O)R¹⁸, —S(═O)₂R¹⁸, —S(═O)₂NH₂,
wherein R¹⁸ and R¹⁹ which may be the same or different independently are selected from hydrogen and C₁₋₆-alkyl or R¹⁸ and R¹⁹ together with the nitrogen atom to which they are attached form a 3 to 8 membered cyclic ring optionally containing one or two further heteroatoms selected from oxygen, sulfur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkylcarbonyl, heteroaroyl, C₃₋₈-heterocyclylcarbonyl, —O-aryl, —O—C₃₋₈-cyclo-alkyl, —O-heteroaryl, —O—C₃₋₈-heterocyclyl, —S-aryl, —S—C₃₋₈-cycloalkyl, —S-heteroaryl, —S—C₃₋₈-heterocyclyl,
which may optionally be substituted with one to three substituents selected from hydroxy, halogen, cyano, nitro, —NR²⁰R²¹, —C(═O)NR²⁰R²¹, —OC(═O)NR²⁰R²¹, —OCH₂C(O)NR²⁰R²¹, C₁₋₆-alkyl, C₂₋₆-alkenyl, C₂₋₆-alkynyl, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R² , —S(═O)₂NH₂,
wherein R²⁰ and R²¹ which may be the same or different independently are selected from hydrogen and C₁₋₆-alkyl or R²⁰ and R²¹ together with the nitrogen atom to which they are attached form a 3 to 8 membered cyclic ring optionally containing one or two further heteroatoms selected from oxygen, sulfur and nitrogen.

46. A compound according to claim 45 wherein D is C₃₋₈-cycloalkyl, aryl or heteroaryl, which are optionally substituted with one or more substituents selected from halogen, C₁,-alkyl, and phenyl-C₁₋₆-alkoxy.

47. A compound according to claim 45 wherein D is
hydroxy, —NR¹²R¹³, C₁₋₆-alkoxy, —C(═O)OR¹², —CF₃, C₁₋₆-alkyl,
wherein R¹² and R¹³ which may be the same or different independently are selected from hydrogen and C₁₋₆-alkyl or R¹² and R¹³ together with the nitrogen atom to which they are attached form a 3 to 8 membered cyclic ring optionally containing one or two further heteroatoms selected from oxygen, sulfur and nitrogen,
aryl, C₃₋₈-cycloalkyl, heteroaryl, C₃₋₈-heterocyclyl, aryl-C₁₋₆-alkoxy,
which may optionally be substituted with one to three substituents selected from
hydroxy, halogen, cyano, nitro, —NR¹⁶R¹⁷, —C(═O)NR¹⁶R¹⁷, —OC(═O)NR¹⁶R¹⁷, —OCH₂C(═O)NR¹⁶R¹⁷, C₁₋₆-alkoxy, —C(═O)OR¹⁶, —C(═O)R¹⁶, —NHC(═O)R¹⁶, —CHF₂, —CF₃, —OCF₃, —OCHF₂, —OCH₂CF₃, —OCF₂CHF₂, —SCF₃, —SR¹⁶, —S(═O)R¹⁶, —S(═O)₂R¹⁵, —S(═O)₂NH₂,
wherein R¹⁶ and R¹⁷ which may be the same or different independently are selected from hydrogen and C₁₋₆-alkyl or R¹⁶ and R¹⁷ together with the nitrogen atom to which they are attached form a 3 to 8 membered cyclic ring optionally containing one or two further heteroatoms selected from oxygen, sulfur and nitrogen,
C₁₋₆-alkyl, C₂₋₆-alkenyl, C₂₋₆-calkynyl,
which may optionally be substituted with one or two substituents selected from hydroxy, halogen, cyano, nitro, —NR¹⁸R¹⁹, —C(═O)NR¹⁸R¹⁹, —OC(═O)NR¹⁸R¹⁹, —OCH₂C(═O)NR¹⁸R¹⁹, C₁₋₆-alkoxy, —C(═O)OR¹⁸, —C(═O)R¹⁸, —NHC(═O)R¹⁸, —CHF₂, —CF₃, —OCF₃, —OCHF₂, —OCH₂CF₃, —OCF₂CHF₂, —SCF₃, —SR¹⁸, —S(═O)R¹⁸, —S(═O)₂R¹⁸, —S(═O)₂N H₂,
wherein R¹⁸ and R¹⁹ which may be the same or different independently are selected from hydrogen and C₁₋₆-alkyl or R¹⁸ and R¹⁹ together with the nitrogen atom to which they are attached form a 3 to 8 membered cyclic ring optionally containing one or two further heteroatoms selected from oxygen, sulfur and nitrogen,
aryl, C₃₋₈-cycloalkyl, heteroaryl, C₃₋₈-heterocyclyl, aryl-C₁₋₆-alkyl, C₃₋₈-cycloalkyl-C₁₋₆-alkyl, heteroaryl-C₁₋₆-alkyl, C₃₋₈-heterocyclyl-C₁₋₆-alkyl, aryl-C₁₋₆-alkoxy, C₃₋₈-cycloalkyl-C₁₋₆-alkoxy, heteroaryl-C₁₋₆-alkoxy, C₃₋₈-heterocyclyl-C₁₋₆-alkoxy, aroyl, C₃₋₈-cycloalkylcarbonyl, heteroaroyl, C₃₋₈-heterocyclylcarbonyl, —O-aryl, —O—C₃₋₈-cyclo-alkyl, —O-heteroaryl, —O—C₃₋₈-heterocyclyl, —S-aryl, —S-C₃₋₈-cycloalkyl, —S-heteroaryl, —S-C₃₋₈-heterocyclyl,
which may optionally be substituted with one to three substituents selected from hydroxy, halogen, cyano, nitro, —NR²⁰R²¹, —C(═O)NR²⁰R²¹, —OC(═O)NR²⁰R²¹, —OCH₂C(═O)NR²⁰R²¹, C₁₋₆-alkyl, C₂₋₆-alkenyl, C₂₋₆-alkynyl, C₁₋₆-alkoxy, —C(═O)OR²⁰, —C(═O)R²⁰, —NHC(═O)R²⁰, —CHF₂, —CF₃, —OCF₃, —OCHF₂, —OCH₂CF₃, —OCF₂CHF₂, —SCF₃, —SR²⁰, —S(═O)R²⁰, —S(═O)₂R²⁰, —S(═O)₂NH₂,
wherein R²⁰ and R²¹ which may be the same or different independently are selected from hydrogen and C₁₋₆ alkyl or R²⁰ and R²¹ together with the nitrogen atom to which they are attached form a 3 to 8 membered cyclic ring optionally containing one or two further heteroatoms selected from oxygen, sulfur and nitrogen.

48. A compound according to claim 47 wherein D is
hydroxy, —NR¹²R¹³, C₁₋₆-alkoxy, —C(═O)OR¹², —CF₃, C₁₋₆-alkyl,
wherein R¹² and R¹³ which may be the same or different independently are selected from hydrogen and C₁₋₆-alkyl,
cyclopropyl, phenyl, naphthyl, morpholino, pyridinyl, tetrahydropyridinyl, thiophenyl, benzothiophenyl, phenyl-C₁₋₆-alkoxy,
which may optionally be substituted with one to three substituents selected from
hydroxy, halogen, nitro, —NR¹⁶R¹⁷, C₁₋₆-alkoxy, —CF₃, —OCF₃, C₁₋₆-alkyl,
wherein R¹⁶ and R¹⁷ which may be the same or different independently are selected from hydrogen and C₁₋₆-alkyl,
phenyl, phenyl-C₁₋₆-alkoxy,
which may optionally be substituted with one to three substituents selected from halogen, nitro, —NR²⁰R²¹, C₁₋₆-alkoxy, —CF₃, —OCF₃, C₁₋₆-alkyl,
wherein R²⁰ and R²¹ which may be the same or different independently are selected from hydrogen and C₁₋₆-alkyl.

49. A compound according to claim 48 wherein D is
hydroxy, —NR¹²R¹³, C₁₋₆-alkoxy, —C(═O)OR¹², —CF₃, C₁₋₆-alkyl,
wherein R¹² and R¹³ which may be the same or different independently are selected from hydrogen and C₁₋₆-alkyl,
cyclopropyl, phenyl, naphthyl, morpholino, pyridinyl, thiophenyl, benzothiophenyl, phenyl-C₁₋₆-alkoxy,
which may optionally be substituted with one to three substituents selected from
hydroxy, halogen, nitro, —NR¹⁶R¹⁷, C₁₋₆-alkoxy, —CF₃, —OCF₃, C₁₋₆-alkyl,
wherein R¹⁶ and R¹⁷ which may be the same or different independently are selected from hydrogen and C₁₋₆-alkyl,
phenyl, phenyl-C₁₋₆-alkoxy,
which may optionally be substituted with one to three substituents selected from halogen, nitro, —NR²⁰R²¹, C₁₋₆-alkoxy, —CF₃, —OCF₃, C₁₋₆-alkyl,
wherein R²⁰ and R²¹ which may be the same or different independently are selected from hydrogen and C₁₋₆-alkyl.

50. A compound according to claim 48 wherein D is
cyclopropyl, phenyl, pyridinyl, tetrahydropyridinyl, thiophenyl,
which may optionally be substituted with one to three substituents selected from
hydroxy, halogen, nitro, —NR¹⁶R¹⁷, C₁₋₆-alkoxy, —CF₃, —OCF₃, C₁₋₆-alkyl,
wherein R¹⁶ and R¹⁷ which may be the same or different independently are selected from hydrogen and C₁₋₆-alkyl,
phenyl, phenyl-C₁₋₆-alkoxy,
which may optionally be substituted with one to three substituents selected from halogen, nitro, —NR²⁰R²¹, C₁₋₆-alkoxy, —CF₃, —OCF₃, C₁₋₆-alkyl,
wherein R²⁰ and R²¹ which may be the same or different independently are selected from hydrogen and C₁₋₆-alkyl.

51. A compound according to claim 50 wherein D is cyclopropyl, phenyl, pyridinyl, tetrahydropyridinyl or thiophenyl, which are optionally substituted with one to three substituents selected from halogen, C₁₋₆-alkyl and phenyl-C₁₋₆-alkoxy.

52. A compound according to claim 51 wherein D is cyclopropyl or pyridinyl.

53. A compound according to claim 37 wherein E is C₁₋₆-alkyl, C₁₋₆-alkoxy or —C(═O)O—C₁₋₆-alkyl.

54. Use of a compound according to any one of the preceding claims 34 to 53 as a pharmaceutical composition.

55. A pharmaceutical composition comprising, as an active ingredient, at least one compound according to any one of the claims 34 to 53 together with one or more pharmaceutically acceptable carriers or excipients.

56. A pharmaceutical composition according to claim 55 in unit dosage form, comprising from about 0.05 mg to about 1000 mg, preferably from about 0.1 mg to about 500 mg and especially preferred from about 0.5 mg to about 200 mg of the compound according to any one of the claims 34 to 53.

57. A method for the treatment and/or prevention of diseases and disorders wherein an inhibition of GSK-3 is beneficial the method comprising administering to a subject in need thereof an effective amount of a compound according to any one of the claims 1 to 19 or 34 to 53 or a pharmaceutical composition according to claim 55 or 56.

58. The method according to claim 57 wherein the effective amount of the compound is in the range of from about 0.05 mg to about 2000 mg, preferably from about 0.1 mg to about 1000 mg and especially preferred from about 0.5 mg to about 500 mg per day.
